(19) United States
(12) Patent Application Publication
(10) Pub. No.: US 2021/0209963 A1
(43) Pub. Date: Jul. 8, 2021

(54) System, Method, Process and Apparatus for Assisting in Formulating and Attaining Healthy Weight Management Goals

(71) Applicant: The Engineered Non-Diet, LLC, Fayetteville, TX (US)

(72) Inventors: Tom H. Hill, Fayetteville, TX (US); Thomas E. Mueller, Fayetteville, TX (US); Carlos A. Torres, Montgomery, TX (US); Reagan D. Brown, Fayetteville, TX (US)

(21) Appl. No.: 16/961,068

(22) PCT Filed: Jan. 24, 2019
(86) PCT No.: PCT/US2019/014963

§ 371 Date: Jul. 9, 2020

Related U.S. Application Data
(60) Provisional application No. 62/621,282, filed on Jan. 24, 2018.

(51) Int. Cl. G09B 19/00; G16H 20/60; A23L 33/00; A23L 33/17; A23L 33/21

(52) U.S. Cl. CPC G09B 19/0092; G16H 20/60; G06K 7/1413; A23L 33/17; A23L 33/21; A23L 33/30

(57) ABSTRACT

A base rating number calculation determines a number rating providing a practical tool for assisting in the selection of foods best suited for healthy weight management. The rating tool is a component of a comprehensive health plan including food quality rating, formulating healthy weight management goals, calculating and reporting on progress in attaining goals and presenting informative and motivational visual displays illustrating the status of goal attainment.

### CROSS REFERENCE TO RELATED APPLICATION

This application claims priority to U.S. Application No. 62/621,282 filed on Jan. 24, 2018 the disclosure of which is incorporated herein by reference for all purposes.

### FIELD OF THE INVENTION

The present invention relates generally to the evaluation, selection and consumption of foods in a healthy weight management program and the implementing of computer applications for monitoring, recording, processing and reporting user progress in attaining the goals of the program.

### BACKGROUND OF THE INVENTION

In May of 2012, a systematic review of the evidence on Calorie density entitled Dietary Energy Density and Body Weight in Adults and Children: A Systematic Review, incorporated herein by reference for all purposes, was published in the Journal of the Academy of Nutrition and Dietetics that concluded “ . . . there is strong and consistent evidence in [adults showing] that consuming a diet higher in [Calorie] density is associated with increased body weight, while consuming a diet that is relatively low in [Calorie] density improves weight loss and weight maintenance.” In addition, it also found that diets low in Calorie density are also automatically higher in nutrient density and overall diet quality.

The study noted further:

“Overall, our findings highlight the crowing body of scientific evidence suggesting a relationship between energy density and body weight in adults, children, and adolescents, such that consuming diets lower in energy density may be an effective strategy for managing body weight. However, there is a need for more effective public health strategies targeting all segments of the US population to better communicate what energy density means and how it is associated with body weight, how to estimate energy density for different products based on food label information, how to decrease the dietary energy density by following key recommendations of the 2010 DGA, and how to sustain weight loss benefits for lower-energy-density diets in the long term.”

Implementation of the findings of this study can be difficult for the ordinary consumer. Even those consumers informed of the benefits of consuming foods that are relatively low in Calorie density are faced with the task of selecting foods that meet the criteria. This selection process can be complicated and difficult, stifling incentive and discouraging motivation to follow a healthy diet program based on the accepted principle of consuming nutrient rich foods with low Calorie densities.

A 2016 paper entitled Link Between Food Energy Density and Body Weight Changes in Obese Adults, incorporated herein by reference for all purposes, published in PMC US National Library of Medicine, National Institutes of Health, based on a study of a number of earlier studies and clinical trials concluded:

“Dietary approaches, based on energy density of foods, show strong evidence that it could reduce body weight and prevent weight regain. The arguments for such an approach come from experimental studies showing that the total amount of food is the driving force for satiety, and thus, the intake of low energy dense foods leads to a reduction in energy intake in obese subjects. In clinical practice it will be possible without high efforts to consult for a diet including high amounts of low energy dense foods that will result in a successful weight loss strategy . . . ”

The above-cited Journal of the Academy of Nutrition and Dietetics concluded that practical consulting material is still missing to help practitioners applying this approach. In addition, simple to apply assessment tools of food energy density could be advantageous.

The article points out the need for a simple assessment tool for applying dietary approaches recognizing that total amount of food is the driving force for satiety, and thus, the intake of low energy dense foods leads to a reduction in energy intake in obese subjects.

A difficulty in benefiting from prior art diet and health programs, including proposed programs such as called for in the two studies above, is that the user has no simple tool for implementing the information about food nutrient content to assist in making good food choices.

It is an important object of the present invention to better communicate what food energy density means and how it is associated with body weight, how to estimate energy density for different products based on food label information and how to sustain weight loss benefits for lower-energy-density diets in the long term.

The prior art includes a number of methods that analyze food to develop rating or numbering systems intended to help users eat so as to reach desired weight goals. These methods can be very complex, requiring multiple calculations involving a relatively large number of food component variables.

While these prior art methods may accurately rank or analyze foods, they can be complicated and tedious, discouraging their implementation and continued use. Importantly, some of these prior art methods and ratings overlook the importance of the nutritional content of the food they rate, focusing instead on total Calories consumed.

The teachings of U.S. Pat. Nos. 6,040,531 and 7,620,531, both incorporated herein by reference for all purposes, employ algorithms that consider dietary fiber density in calculating the values used in their food rating systems and both use calculations to factor the algorithm results to number values that may be more easily remembered by the dieter. Both use multi-step calculations and include a number of food components in rating or analyzing each food. Neither patent provides a method for rating food that is comprehensive, accurate and also simple to calculate, understand and implement.

U.S. Pat. 6,040,531 teaches the use of the rating in a point accumulation program designed to meet a dieter's goals. The point accumulation is applied to a daily point goal range allotment that is selected from a table based upon the user's physical condition and goals. The so-called POINTS are based on the Calories fat and dietary fiber content of the serving wherein each numerical point value is based on a predetermined fraction of the energy content of the food serving increased on the basis of fat content and decreased on the basis of fiber content.

Regardless of the basic principles underlying the program, if the program involves rating food, the user should understand and have answers as to how a specific food meets the user's current weight management requirements. It should also show how the rating value assists in food selection and how the rating evaluates the aggregated nutritional attributes of food already consumed. Moreover, the user should know how the aggregated attributes of the food consumed, and being considered for consumption, bear on the specific nutritional and weight management requirements and goals of the individual user. The information should be easy to obtain, accurately presented and easy to understand and implement.

U.S. Pat. No. 7,620,531 to Johnson uses the nutrient values in the food and two formulas to calculate two rating numbers referred to as a Fullness Factor ((FF) and a Nutritional Density Rating (NDR)). Johnson describes a method of assisting individuals in making food choices by providing two nutritional indices. One is a numerical expression of a foods overall nutrient density and the other is a separate numerical expression that relates to a food's caloric density that is a prediction of the satiating effect of a food. A visual aid in the form of a graph or chart is provided so individuals using the indices can refer to the chart to determine food selections in accordance with the individual's dietary goals. A third calculation is used to determine a food's rating with regard to satiety and a forth calculation is used to determine a food's rating with regard to nutritional density.

Johnson's Fullness Factor is calculated with a formula developed using a multivariate analysis of existing data to predict Satiety from the nutrient content of a given food or recipe. The calculation yields Fullness Factor values that fall within the range of 0 to 5. Foods with high Fullness Factor values are more likely to satisfy an individual's hunger with fewer Calories. Foods with low Fullness Factor values are less likely to satisfy hunger. Johnson shows an illustration comparing the calculated Fullness Factor values with the satiety values obtained during a 1995 experimental study, entitled A Satiety Index of Common Foods, incorporated herein by reference for all purposes, (1995 Study), Johnson notes that the Fullness Factor calculation provides an estimate of food satiety prior to consumption.

To the extent that the fullness factor has importance in determining the quality and suitability of the food for a healthy weight management program, the benefit of that information is included into the Johnson rating calculations through a complex algorithm.

The two formulas used in calculating the Johnson Fullness Factor and Nutritional Density Rating numbers are lengthy and complex as illustrated by the following listing. Complete nutritional information for a food may require up to 4 separate calculations.

The Johnson formula for calculating the Fullness Factor includes:
1. A function that returns the maximum of either X or Y;
2. A function that returns the minimum of either X or Y;
3. Total Calories per hundred grams serving of the food;
4. Grams of protein per hundred grams of the food;
5. Grams of total fat per hundred grams of the food;
6. 7 constants;
7. 2 coefficients; and
8. 2 exponential powers.
The Johnson formula for calculating the Nutrient Density Rating includes:
1. A function that returns the natural-based log of the number X;
2. A function that returns the maximum of either X or Y;
3. A function that returns the minimum of either X or Y;
4. The amount of given nutrients in the food and the amount of the nutrient specified as a daily value by the US FDA, This is a list of approximately 50 different nutrient quantitative values.
5. The number of Calories in a serving derived from sugar and alcohol;
6. The total number of Calories in a serving;
7. 6 constants;
8. 27 coefficients.

### SUMMARY OF THE INVENTION

The present invention is a weight management system that assists in the attainment of healthy weight management objectives and goals tailored to the specific requirements of the individual. The system is built around a food rating tool that rates the quality of food for its value in the weight management program. In the basic form of the invention, the calculated basic food rating number is graded and rated by the presence of specific nutrients in the food that are desirable for certain health and weight management objectives.

In its simplest implementation, users of the system are directed to use the food rating tool to identify the most desirable foods for maintaining a healthy weight management program. The general directive is to preferentially select foods with the highest rating numbers.

In one form of the invention, a special programming application (Application or App) is incorporated into a smart phone to calculate a food rating value consisting of a single number (One Number) having a value that is factored by the presence of specific nutrients in the food. Food for individuals with a specific health or weight management concern will have differing One Number food ratings. For example, boiled shrimp has a high One Number rating for the average healthy person wishing only to maintain weight. The One Number rating for a person who has a cholesterol concern is much lower.

The computer and Application of the present invention implement a healthy weight management plan for the individual user built around the One Number rating tool. The special needs of the user are programmed into the Application to formulate the weight management plan and the computer is used to receive input information regarding food nutrients and activity energy burn. Nutrient data may also be supplied by a barcode scanner. Databases containing nutrient information are carried in the computer memory and may also be obtained through Internet access to the Cloud. The input information is processed by the Application in the computer to provide current data and visual displays regarding status of attainment of the objectives and goals formulated in the weight management plan.

The computer provides multiple single screen visual displays of the current One Number rating of the historical status of the aggregate cumulative quality of the food consumed and the amount of weight gained or lost during the monitored period. Weight changes are displayed as physical tablespoons of body fat to personalize and incentivize the user. A food may be tested for its effect on body fat change and the cumulative One Number rating value for food already consumed, Recommended Daily Nutrient Allowances (RDA's) are tailored to the user. The visual displays also show such information as current and historical status of the amounts of nutrients consumed, dietary balances and ratios, and net energy gain or deficit.

Weight change due to activity energy burn is calculated and displayed using an algorithm that produces significant accuracy without using data produced by a wrist mounted heart rate monitor. The predicted change in weight is reported in real time, minute-by-minute, in small step number increments. User estimated information may be used to calculate activity energy burn when no monitor information is provided.

The present invention addresses the recognized need for a simple assessment tool for applying dietary approaches recognizing that total amount of food is the driving force for satiety, and thus, the intake of low energy dense foods leads to a reduction in energy intake in obese subjects. The computer device and tool of the present invention provide visual displays that assist in formulating a healthy weight management program, and in formulating, guiding and motivating compliance with the objectives of the program.

These and further features and advantages of the present invention will become apparent from the following detailed description, wherein reference is made to the figures in the accompanying drawings.

### BRIEF DESCRIPTION OF THE DRAWINGS

FIG. 1 is a graphical illustration of a prior art calculation of fullness factor versus the calculation of the present invention.

FIG. 2 illustrates a graph that compares the users actual manually calculated and recorded weight to the theoretical weight calculated by the B4B system of the present invention.

FIG. 3 is a graph that compares the user's actual weight to the theoretical weight calculated by the user's conventional wrist mounted fitness tracker (the tracker).

FIG. 4 lists the 3 primary algorithms for the basic B4B /calculation.

FIG. 5 illustrates a fiber rating system of the present invention.

FIG. 6 illustrates a cholesterol grading system of the present invention.

FIG. 7 illustrates a personalized unit of weight measurement of the present invention called a TUF.

FIG. 8 illustrates a derivation of the TUF energy unit.

FIG. 9 is a portion of a spreadsheet illustrating a nutrient database.

FIG. 10 is another portion of the spreadsheet of FIG. 9 illustrating a nutrient database.

FIG. 11 is another portion of the spreadsheet of FIG. 9 illustrating a nutrient database.

FIG. 12 is another portion of the spreadsheet of FIG. 9 illustrating a nutrient database.

FIG. 13 is another portion of the spreadsheet of FIG. 9 illustrating a nutrient database.

FIGS. 14-39 are screenshots taken from a smartphone operating the App of the present invention.

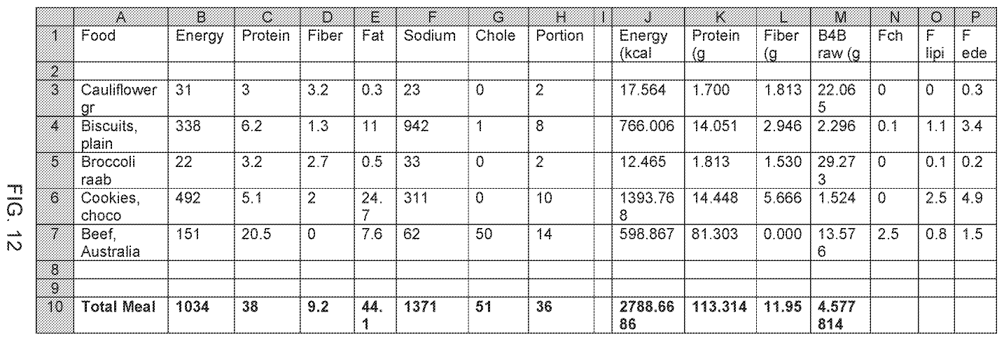

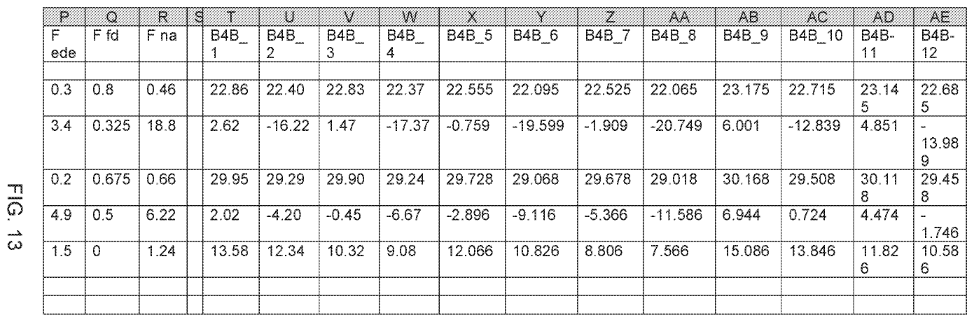

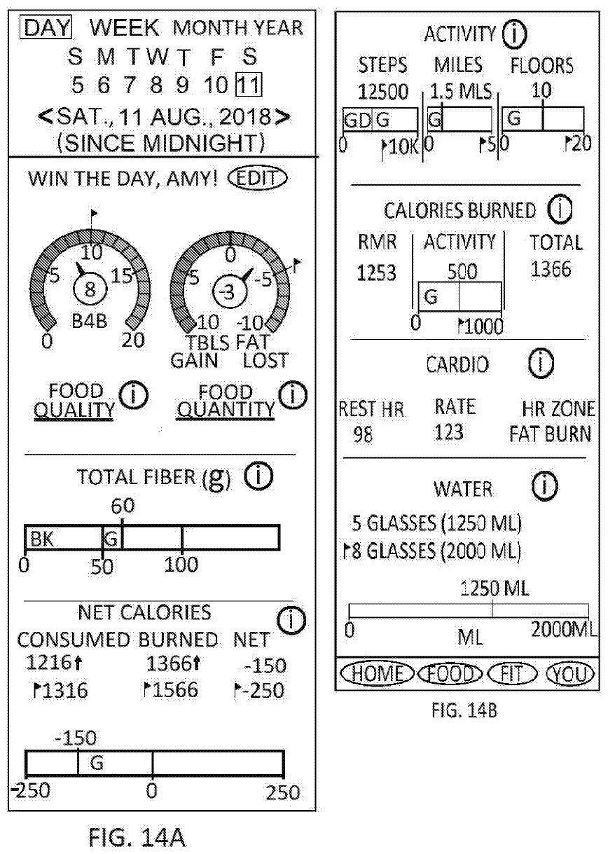

In FIGS. 14-36, shaded color areas are indicated by the following abbreviations: BK=black, BL=blue, GD=gold, G=green, O=orange, and P =purple. A single large X crosshatched area indicates consumed nutrients in the bar graphs.

### DETAILED DESCRIPTION OF PREFERRED EMBODIMENTS

The present invention is a tool for formulating and implementing a healthy weight management plan. A most basic form of the present invention includes a tool for calculating a rating number for a plurality of foods derived from each food's energy, protein and fiber content, then rating the quality of foods relative to one another by the value of the calculated number wherein foods with the highest number have the highest quality. The calculated rating number is further graded and ranked as a function of specific additional nutrients in the food to address specific nutritional requirements of individuals.

The basic tool of the present invention is called “Bang for the Buck” (B4B). As used herein, the term B4B is used to identify the System, Method, Process, and Apparatus of the present invention and its related variations and components.

The B4B system provides a simple and accurate food rating tool that may be easily implemented in the formulation and attainment of individual healthy weight management goals.

The superiority of the B4B system, in terms of its simplicity and accuracy, is evidenced by comparing it with prior art food rating systems such as taught in the Johnson patent.

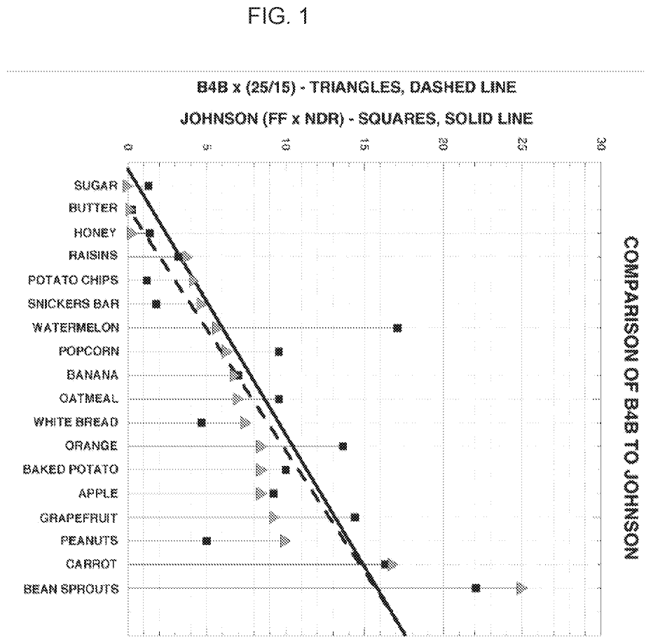

FIG. 1 illustrates the correlation of satiety predictions using the B4B system with the results of the 1955 experimental study and the predictions made by the Johnson Fullness Factor calculation. The satiety calculation is only one of the two major calculations required in the Johnson teaching.

The graph for the B4B rating number calculations was created by multiplying the calculated B4B values by 1.67 to center the Johnson and B4B data sets so they would approximately overlay. In order to get Johnson's rating on a comparable basis for the chart of FIG. 1, Johnson's FF values have been multiplied with Johnson's NDR values.

Several of the foods listed by Johnson have been omitted from the display in FIG. 1. since there is no way to quantify their attributes, either for energy density, nutrient density or what Johnson calls Fullness Factor. For example, spaghetti, without specifying the complete list of ingredients in the sauce, there is no way to judge what these attributes will be.

The following may be noted from the comparison shown in FIG. 1:
The two computer-generated best fit trend lines almost exactly overlay. This indicates very good overall correlation between Johnson's rankings and the B4B rankings for the foods Johnson listed that had known attributes.

For individual foods, except watermelon, rankings were similar. The data points even touched one another on more than 20 percent of the comparisons. For watermelon, there is sufficient reason to have more confidence in the B43 value than Johnson's value, as the B4B value represents the ratio of nutrient density to energy density, where Johnson's value is based on a subjective feeling of “fullness” reported by 12 test subjects. It is not plausible that watermelon is nutritionally superior to everything on the list except bean sprouts, “Fullness” notwithstanding.

The same information regarding satiety reported in the 1995 Study and determined by Johnson is obtained from the simple, extremely less complex calculations of the B43 system of the present invention. The complexity of the Johnson calculations is detailed in the previous discussion of prior art.

In addition to the complexity involved in calculating the Johnson ratings, implementing the knowledge obtained from such calculations can be problematic. While the steps involved in positioning a food on Johnson's graphical representation using the Fullness Factor and Nutritional Density Rating as indices may be performed by suitable computing means, the final step in implementing the method requires the user to interpret the graphical presentation to determine suitability of the food for meeting the user's goals. This latter requirement imposes subjectivity and resulting uncertainty in the user's decision-making. Even where multiple foods are being compared on a single graph so that the points for the food can be seen to be at obviously different locations, uncertainty can exist regarding the meaning of the position on the graphical representation where the user has multiple dietary requirements.

Another distinction between the B4B system and the Johnson teaching is that in the Johnson system the same calculated number and the same plotted point location for any given food are used for decision making by all users of the method, regardless of their individual nutritional needs. Users that do not qualify as average healthy people do not necessarily receive the best guidance from the

Johnson calculation. For example, in the Johnson system the food for a person with high blood pressure is given the same graphical position or numerical rating for the food as that calculated for the average person.

### Measurement and Calculation of Activity Energy Burn

Many commonly used prior art weight management systems monitor activity energy burn, in addition to the normal RMR (Resting Metabolic Rate) energy burn, to calculate a net Calorie gain or loss over the monitored time period, The results of the calculation can be incorrect when the activity monitoring devices inaccurately measure the user's activity or the algorithm used to calculate the weight change information is faulty.

### Daily Scale Weight vs Calculated Weight

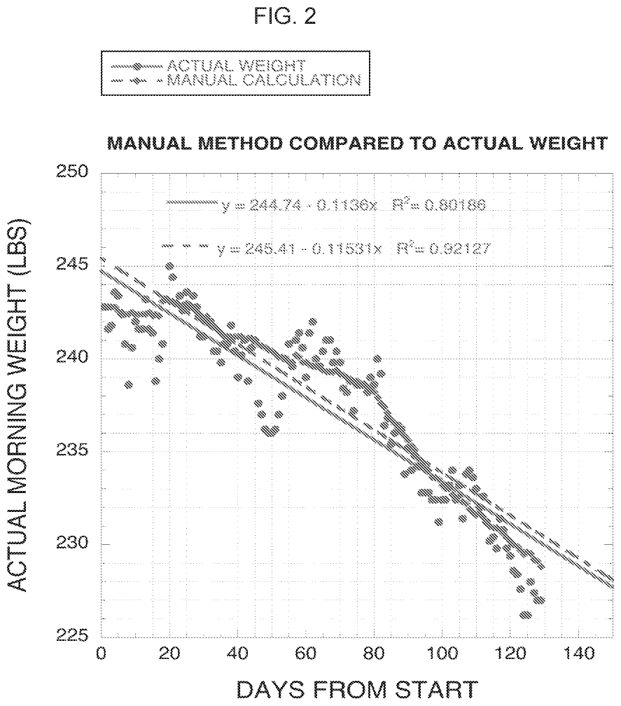

The circles on the graph of FIG, 2 represent the user's actual daily weight as measured on a weight scale. The squares (diamonds) on the graph of FIG. 2 represent the user's calculated predicted weight using the equations of the Engineer's non-Diet shown in the attached Calculations and Data sheet.

The two data recordings are correlated to determine their closeness. The straight brown line (the uppermost line) is the computer-generated linear curve from the brown data points (squares). The straight red line (the bottom line) is the computer-generated linear curve from the green data points (circles). The Y and R notations on the graph are the slope intercept information of the best fit for the corresponding colored lines.

The two lines almost exactly overlap. The scatter of the green dots indicates the error in using daily weight as an indication of what is happening in a diet plan.

Some prior art devices also measure and use heart rate to calculate the activity energy burn. The systems that use heart rate as a factor in calculating the activity energy burn are subject to producing relatively large errors in the calculated weight change if the heart rate information is inaccurate or the algorithm used to calculate the weight change using the heart rate information is faulty.

A possible reason for the degree of the inaccuracies is that the heart rate calculation factor is capable of causing an error that is greater than the inaccuracy attributable to ignoring heart rate measurement as a part of the calculation. Stated differently, it is likely that the inaccuracy of the calculated result where heart rate is a component of the formula for the energy burn calculation is greater than the lack of precision that might arise from the absence of the heart rate component in the calculation.

The possible inaccuracy of wrist carried systems that use heart rate monitoring as a part of the energy burn calculation is well documented in the prior art. Recently introduced devices have made improvements in the accuracy of measuring and calculating heart rate associated energy burn, however the possibility of error in either measurement or calculation of the heart rate associated burn remains present if even the very best devices are inappropriately used or worn or the algorithm used in processing the information is inaccurate.

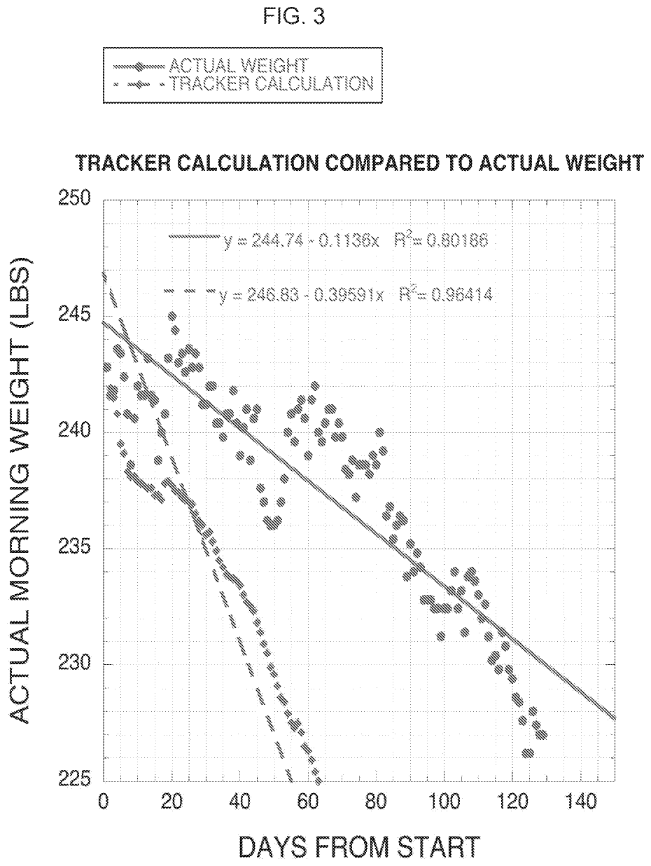

The graph of FIG. 3 compares the user's actual scale measured weight (green circles) to the theoretical weight predicted by the calculations using the equations of the Engineer's non-Diet shown in the attached Calculations and Data sheet and by the Data (Brown squares) calculated by the user's conventional wrist mounted fitness tracker (the tracker).

The user's actual morning weights were taken at the same time and under the same conditions on a repeatable digital scale. As may be seen from the graphs, the fitness tracker calculates that the user is losing weight more than twice as fast as is actually the case. This discrepancy is due to errors in the tracker's wrist heart rate measurement which was repeatedly found to be registering heart rates up to 40 BPM faster than the rate measured manually.

The erroneous heart rate monitor results are consistent with those reported in a published study of several commercial wrist heart rate monitors that confirms their inaccuracy.

Accurate heart rate monitoring is more difficult than accurate physical step monitoring. The heart rate data is a significant component in many conventional automated weight management devices. Because of the significance given the heart rate calculation in the conventional prior art algorithms, any errors in monitoring heart rate erroneously skew the calculated weight change result making the calculated weight change vary significantly from the actual weight change.

The methods of the present invention, using algorithms that require only user physical movement information in the calculation of activity energy burn, consistently produce a more accurate calculated weight change result than more complicated conventional methods and algorithms that use error-prone wrist monitored heart rate information and/or erroneous algorithms in the weight change calculation.

In one form of the invention, the B4B tool is an easy-to-implement and understand, quantitative and qualitative food rating system based primarily on the foods' energy, protein and fiber content. The plan of the present invention is implemented by the selection and consumption of higher-ranked foods as determined by the value of the rating number. The food quality may be further graded and ranked according to other nutrients of particular importance to the user.

By limiting portion size (measured only in Calories in the food eaten, not weight or volume of the food), weight will be lost so long as a cumulative energy deficit is maintained over some extended period.

Food ranking tables created with the rating numbers calculated by the B4B method can improve success in attaining the user's nutrition and weight management goals by providing information identifying food choices with more protein and dietary fiber per Calorie. Both are very important to weight control and general well-being. The primary rule to follow is to choose higher-ranked foods over lower-ranked foods.

There are three separate B4B ratings in the basic B4B system: B4BF (grams of fiber per 100 Calories of food), B4Bₚ (grams of protein per 100 Calories of food), and B4B_{TOTAL} (the sum of the two ratings). For foods that contain one but not both of the two nutrients, the rating for the missing nutrient is zero, so the total rating (B4B_{TOTAL}) will equal the rating for the nutrient that's present.

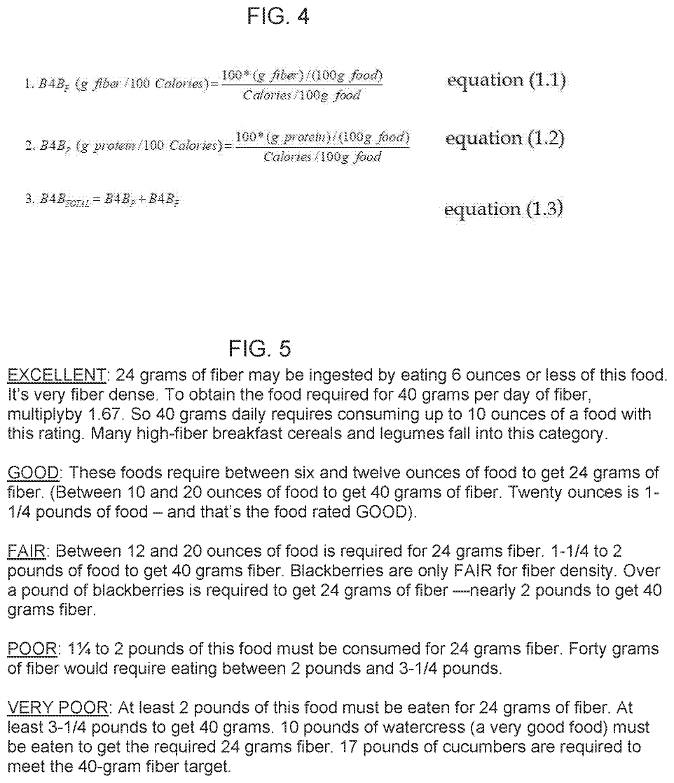

Equations (1.1), (1.2) and (1.3) shown in FIG. 4 are the algorithms for the basic B4B rating system.

Example:

Blackberries have 3.2 g of protein nutrient per hundred Calories (B4B_{P}) and 12.3 g of fiber nutrient per hundred Calories (B4B_{F}). The calculated and tabulated B4B_{TOTAL} (shown in the rating tables) is 15.6 (the discrepancy in the total of the individual items versus the total displayed is due to the rounding performed on each of the individual item values).

### The Fiber Density Rating

Fiber is a very important dietary commodity that few get in sufficient amounts. It is generally recommended that the average person eat 24 to 40 grams of fiber daily- depending on gender (men need more) and depending on which expert is consulted. Yet Americans get less than one-third to one-half of what is needed, and suffer weight gain and health problems as a result. The deficit exists, in part, because it is often impractical to eat enough food to get the recommended minimum daily fiber dose.

A major reason we don't get enough fiber is a problem resulting from the ‘low-fiber density’ of the foods commonly in our diet. Fiber is present in many fiber-containing foods that are low in Calories and nutritionally good for us. The unit of fiber density is grams of fiber per total weight (ounce or pound) of food. On one hand, it's excellent news that many fiber-containing foods are very low in Calories, so a lot of them may be eaten without getting fat. They also tend to be full of vitamins and minerals.

To identify the fiber sources that don't require eating so much of to get a daily minimum, one form of the present invention includes a Fiber Density Rating for the fiber content of the food. See FIG. 5 for the fiber ranking system of the present invention.

Over half of all vegetables rank VERY POOR for fiber density. Less than three percent (3%) rank EXCELLENT.

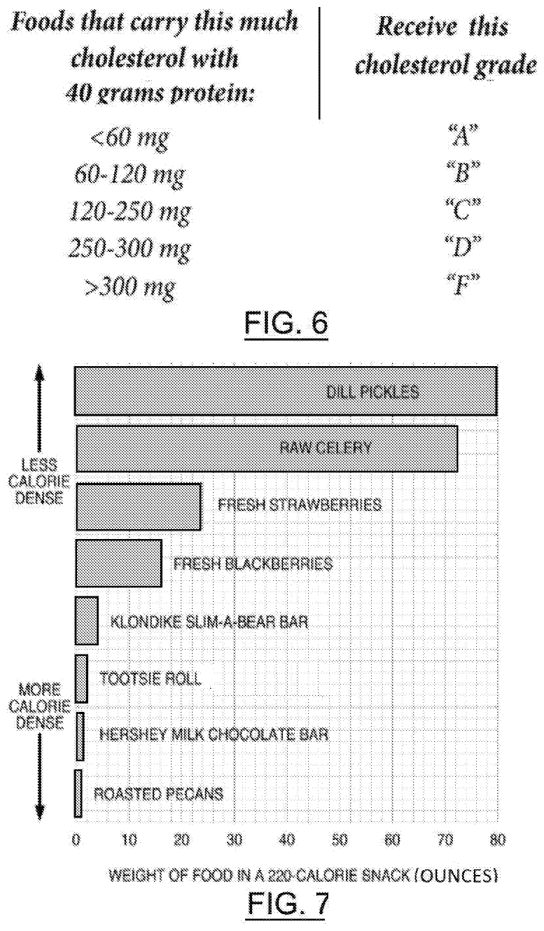

FIG. 6 shows the cholesterol grading system of the present invention. Cholesterol in our blood has long been thought to be a factor in coronary disease risk, hence we make great efforts to reduce our blood cholesterol levels. To grade foods for cholesterol, a scale based on the USDA recommended daily maximum cholesterol limit of 300 milligrams (mg) was used with this reasoning: One needs at least 40 grams of protein daily, (which is often obtained from meat, fish or dairy). If a person consumes just enough of one of these foods to get 40 grams of protein, how much cholesterol would that person have gotten along with the protein?

Summing up all the above, assume one wants to determine the nutritional content per Calorie and cholesterol grade for microwaved bacon. Consult the Pork and Pork Products table (Table 1. example below). Note that microwaved bacon has 135 Calories per ounce, and 8.2 grams protein per 100 Calories. If enough microwaved bacon is eaten to get 40 grams of protein (about 5 ounces—650 Calories more or less), between 60-120 mg cholesterol will have been consumed—not bad, a “3” grade. However, notice that Canadian Bacon has 60% more protein per Calorie (B4B_{P}) than micro-waved bacon. The Canadian

Bacon is a better choice. The 40 grams of protein will be consumed eating only a little over 3 ounces of Canadian bacon (about 170 Calories).

TABLE 1
Example Use of Food Ranking Table
(grams nutrient/100 Calories)

| Pork & Pork Products | CALORIES/ | PROTEIN | FIBER | TOTAL | CHOLESTEROL |
|---|---|---|---|---|---|
| ARRANGED ALPHABETICALLY | oz food | B4B_{P} | B4B_{F} | B4B_{TOTAL} | GRADE |
| Bacon, baked | 155.4 | 6.5 | 0 | 6.5 | B |
| Bacon, broiled, pan-fried or roasted | 153.4 | 6.8 | 0 | 6.8 | B |
| Bacon, microwaved | 135.0 | 8.2 | 0 | 8.2 | B |
| Bacon, pre-sliced, pan-fried | 133.5 | 7.3 | 0 | 7.3 | B |
| Bacon, rendered fat, cooked | 254.6 | 0.0 | 0 | 0.0 | F |
| Bacon, unprepared | 118.2 | 3.0 | 0 | 3.0 | C |
| Breakfast strips, cooked | 130.1 | 6.3 | 0 | 6.3 | C |
| Breakfast strips, raw or unheated | 110.0 | 3.0 | 0 | 3.0 | C |
| Cured, Canadian-style bacon, grilled | 52.5 | 13.1 | 0 | 13.1 | B |

The USDA maintains a public database that gives the Calorie and nutritional contents of thousands of foods. It was from here the raw data to calculate the food rating factors for 95% of the foods in the tables was obtained. The remainder was taken from food labels.

The following are some key points to the B4B system of the present invention.

1. Small differences in B4B rating number are not significant. A food that is rated 15 on the B4B scale won't be significantly different than one rated a point or two higher or lower. The bases of the ratings, Calories and nutrients, are subject to wide variations The nutrients in watercress grown in one lake will be different than in the watercress grown in another lake, Many other measurement variables will decrease the precision of the raw data found in the USDA foods database, from which the ratings mostly come.

2. Wide differences are very meaningful however, and even narrow difference are meaningful at the lower end of the B4B ratings. A food that rates 15 on the B4B scale is clearly preferable to one that rates 5, about the same as a 5 beats a 2. The wider two foods are spaced on a table, the greater they differ. Preferentially select a food having the highest available B4B Rating.

3. Eat a variety of different foods, while preferentially selecting the food with the highest available ranking. Many foods with similar ratings will have different arrays of vitamins and minerals.

4. If the B4B rating for a food is not on one of the rating lists, check the food label, then add the grams protein per serving to the grams fiber per serving. Multiply this sum by 100. Divide the total by Calories per serving. The result is B4B_{TOTAL}.

For example: A food having 120 Calories, 10 grams of fiber and 4 grams of protein per serving will have a B4B_{TOTAL} rating calculated by:

(10+4)×100/120=1400/120=11.7 grams nutrient per 100 Calories.

Another food has 80 Calories, 3 grams fiber and 9 grams protein:

(3+9)×100/80=1200/80=15 grams nutrient per 100 Calories.

If only the rating for either fiber or protein is desired, leave out the grams for the nutrient to be ignored.

5. Portion size is the other half of good food choice. It's total Calorie consumption minus total Calorie burn that determines if one is gaining or losing weight. If trying to lose weight, it does no good to switch to a lower Calorie food and compensate by eating more of it. Better nutrition will be achieved with higher B4B ratings, but weight won't be lost unless more Calories are burned than consumed. Avoid large portion sizes.

### Good Nutrition Using the B4B Rating Tool

This topic is more complicated than the simple energy-balance equation for weight control. It's not enough just to be skinnier. The objective is to be skinnier and well nourished. It's not uncommon to find people in the opposite state—fat but malnourished. Research subjects, up to several hundred pounds overweight are often found malnourished because their diets consist mainly of Calorie-dense but nutrient-poor foods like fatty-meat cuts, sugared sodas, potato chips and the like. The objective in this invention is to direct food consumption away from these foods toward those that are nutrient dense but Calorie poor. As a part of this invention, foods are ranked for nutrient-to-Calorie ratio with the objective of guiding food selection in the right nutritional direction while losing weight.

One source for calculated B4B values is The Engineer's non-Diet book authored by Tom Hill and incorporated herein by reference for all purposes, which ranks food in 34 tables grouped according to general categories. Each food group has two associated tables; first with foods arranged alphabetically and second with foods ranked in descending order according to their fiber and protein content per 100 Calories of food (B4B_{TOTAL}).Except for nutritional supplements and protein bars, the numbers on these tables were generated from raw data taken from the USDA foods database.

The rankings showing the B4B caloric density rating may also further rank food as a function of additional nutrient content such that foods with the same B4B Calorie density number may be rated higher or lower according to their content of other desirable or undesirable components. For example, a specialized B4B food rating may be recommended for a user having diabetes such that food with the same B4B rating but less sugar would be rated above the food with the higher sugar content. This may be seen in the tables with regard to cholesterol where foods with the same B4B rating but a higher letter grade rating of cholesterol would be the preferred food for a user with high cholesterol levels.

An example of a benefit of a table ranking food in this manner is that a dietitian can recommend a diet of foods that have a B4B rating of 15 or above and a cholesterol rating of B or above. The user need only consult the table to determine how a food of interest complies with the recommendation. See sample Table 2 taken from the poultry page of the The Engineer's non-Diet book.

TABLE 2
(grams nutrient/100 Calories)

| Poultry | CALORIES/ | PROTEIN | FIBER | TOTAL | CHOLESTEROL |
|---|---|---|---|---|---|
| RANKED BY TOTAL BANG FOR BUCK (B4B_{TOTAL}) | oz food | B4B_{P} | B4B_{F} | B4B_{TOTAL} | GRADE |
| Turkey from whole dark meat, meat only | 31 | 19.7 | 0 | 19.7 | C |
| Chicken, rotisserie, BBQ, breast meat only | 41 | 19.5 | 0 | 19.5 | B |
| Ostrich, outside strip | 34 | 19.5 | 0 | 19.5 | C |
| Ostrich, round | 33 | 19.0 | 0 | 19.0 | C |
| Guinea hen, meat only | 31 | 18.8 | 0 | 18.8 | B |
| Ostrich, inside strip | 36 | 18.7 | 0 | 18.7 | B |
| Ostrich, fan | 33 | 18.6 | 0 | 18.6 | C |
| Chicken, breast, meat only | 32 | 18.6 | 0 | 18.6 | B |
| Quail, breast, meat only | 35 | 18.4 | 0 | 18.4 | B |
| Pheasant, breast, meat only | 38 | 18.3 | 0 | 18.3 | B |
| Chicken, roasting, meat only | 31 | 18.3 | 0 | 18.3 | C |
| Ostrich, outside strip, cooked | 44 | 18.3 | 0 | 18.3 | C |
| Ostrich, oyster, cooked | 45 | 18.1 | 0 | 18.1 | B |
| Chicken, meat only | 34 | 18.0 | 0 | 18.0 | C |
| Ostrich, tenderloin | 35 | 17.9 | 0 | 17.9 | C |
| Pheasant, meat only | 38 | 17.7 | 0 | 17.7 | B |
| Chicken, wing, meat only | 36 | 17.4 | 0 | 17.4 | B |
| Ostrich, oyster | 35 | 17.2 | 0 | 17.2 | C |
| Turkey, gizzard, all classes | 31 | 16.9 | 0 | 16.9 | F |
| Chicken, stewing, light meat, meat only | 39 | 16.9 | 0 | 16.9 | B |
| Chicken, roasting, dark meat, meat only | 32 | 16.6 | 0 | 16.6 | C |
| Pheasant, leg, meat only | 38 | 16.6 | 8 | 16.4 | C |
| Chicken, dark meat, drumstick, meat only | 33 | 16.4 | 0 | 16.4 | C |
| Quail, meat only | 38 | 16.2 | 0 | 16.2 | C |
| Chicken, dark meat, thigh, meat only | 34 | 16.2 | 0 | 16.2 | C |
| Emu, oyster | 40 | 16.2 | 0 | 16.2 | C |
| Duck, wild, breast, meat only | 35 | 16.1 | 0 | 16.1 | C |
| Chicken, dark meat, meat only | 35 | 16.1 | 0 | 16.1 | C |
| Chicken, leg, meat only | 34 | 16.0 | 0 | 16.0 | C |
| Chicken, rotisserie, BBQ, breast meat and skin | 50 | 15.1 | 0 | 15.1 | C |
| Turkey, whole, giblets | 35 | 14.7 | 0 | 14.7 | F |
| Chicken, giblets | 35 | 14.4 | 0 | 14.4 | F |
| Chicken, stewing, meat only | 42 | 14.4 | 0 | 14.4 | B |
| Chicken, roasting, giblets | 36 | 14.3 | 0 | 14.3 | F |
| Goose, domesticated, meat only | 46 | 14.1 | 0 | 14.1 | C |
| Chicken, capons, giblets | 37 | 14.1 | 0 | 14.1 | F |
| Turkey, all classes, breast, meat and skin | 45 | 13.9 | 0 | 13.9 | B |
| Duck, domesticated, liver | 39 | 13.8 | 0 | 13.8 | F |
| Turkey from whole, light meat, meat and skin | 46 | 13.6 | 0 | 13.6 | B |
| Duck, domesticated, meat only | 38 | 13.5 | 0 | 13.5 | C |
| Poultry food products, ground turkey | 42 | 13.3 | 0 | 13.3 | C |
| Chicken, canned, meat only, with broth | 47 | 13.2 | 0 | 13.2 | B |

The tables showing the caloric density may also display a ranking of the food as a function of the foods desirability for a restricted diet such that two similar foods with the same caloric density may be ranked higher according to which has the most components suitable for the diet. For example, column heading listed “Sugar Control” (not illustrated) would list the food with the lowest sugar content above those having the same B4B number but a higher sugar content. For reference purposes, the B4B value may be added as a single column within the same nutritional tables published by the USDA.

The net effect of the B4B calculation is that food selection guided by the results of the calculation drive the user to nutrient dense and Calorie poor foods.

Simply choosing the higher number between alternative foods will automatically provide the best choice among the selection.

The B4B value is a number that varies approximately between 0 and 40. The higher the B4B number, the more desirable the food for purposes of achieving a healthy weight management objective.

Food choice and portion size are two essential elements that manipulate the energy balance equation for weight management. They determine one's Calorie input. There are huge differences in how concentrated the Calories may be in different foods. Some contain a lot of Calories packed in a small amount of food. These are said to be “energy dense.” To lose fat, these foods should be avoided or eaten very sparingly. On the other end of the scale are foods low in Calories per unit of food. Large quantities of these foods can be eaten without consuming large quantities of Calories.

### Illustration of the “TUF” in Implementing the Teachings of the Present Invention

The present invention includes the motivational incentive of measuring weight changes in forms of a readily visible, personalized unit of measurement called the TUF. The “TUF” pronounced “tough,” is the unit of energy used to evaluate those spur-of-the-moment eating choices. TUF stands for “Tablespoon Unit of Fat” It's the number of consumed Calories that'll add one tablespoon of fat to the body. Likewise, if total consumption for the day is Calorie deficient, consuming one TUF will prevent loss of one extra tablespoon of fat.

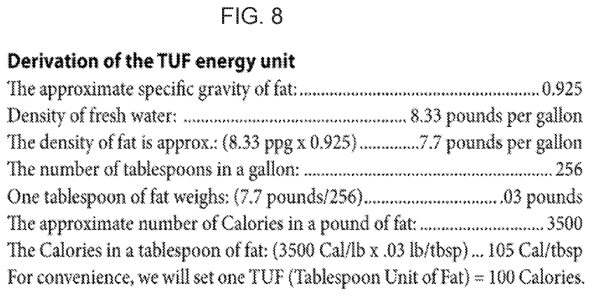

As shown in FIG. 8, one TUF equals about 105 Calories. For ease of calculating and use, the unit is rounded to one-hundred Calories. Now one's choice of foods is clear: a Hershey bar contains two TUFs, more or less, (220 Calories) of energy. Eating it will add (or prevent shedding) two tablespoons of fat. The trade-off is two tablespoons of extra fat for ninety seconds of eating pleasure. The effect of consuming 2 tablespoons of fat is easily personalized by the visualization of this quantity of tangible fat being physically attached to one's body.

Eating one piece of pecan pie will add 600 Calories, six TUF's to one's body. Switching to a positive note, if the day is finished with a six-hundred Calorie energy deficit, six TUF's will have been lost from one's body.

Assume one has 220 Calories available to be consumed in a weight management program. FIG. 7 lists a few choices, any one of which will put about 220 Calories (2 TUFs) into us and, unless burned off, add two tablespoons of fat to us. Our options range from one ounce of roasted pecans to five pounds of dill pickles. These two foods are on opposite ends of the Caloric density scale. Calories are packed into pecans. One can easily eat and an ounce of pecans.

Eating five pounds (about three quarts) of dill pickles in one snack would be very very difficult.

### The Computer Implemented B4B Tool

In one form of the invention, a special programming Application (App) is incorporated into a smart phone to calculate a food rating value comprising a single number having a value that is modified by the presence of specific nutrients in the food and tailored to the special requirements of the individual user. The grading and rating of the number calculated in the basic form of the invention are incorporated into the calculations to produce a “one-number” rating.

The App of the present invention is designed for use preferably with a mobile computing device, such as a smart phone or other suitable computer device. The App is designed to receive and process food nutrition information and physical activity energy burn, perform calculations and otherwise process the received data using and visually displaying cumulative results during the day. The user's historical results are maintained in the computer memory for review to allow the user to assess attainment of program goals. Nutritional data and activity data are obtained from conventional data sources including the US government databases for generic and branded foods, private resources for branded foods and proprietary databases. Activity data may be provided by a separate monitoring device or may be monitored by the computer device itself or may be manually entered by the user based on estimates of activity. The preferred form of the invention provides a major portion of the nutrition data used in the Application in the database of the smart phone computer. Access to cloud-based databanks may be made by VVi-Fi or telephone service.

The App of the present invention addresses the nutritional requirements of the user with regard to several categories of nutrients including one or more selected from a basic category containing protein, fiber, cholesterol, carbohydrates, sugars, fats, saturated fats, added sugars and water. The additional categories include vitamins, minerals, fatty acids and other nutrients of interest.

An important category, and one of the features of the present invention is the balance category that is used to guide the user in selecting proper foods and evaluating the healthfulness of food already consumed and that considered for being consumed. The dietary balance formulas are shown in equations (2.18) through (2.28). See the CALCULATIONS AND DATA listing.

The App of the present invention will also evaluate branded food supplements being consumed by the user by accessing a proprietary database carried in the computer of the mobile device into which the App has been installed.

Where available, information regarding the source of the nutrient, whether animal or vegetable, will be used to evaluate the nutrient.

The App may either use pre-calculated values resulting from the calculations described herein and stored on the onboard database or perform the calculations using the equations when queried by the program.

These calculations fall into two broad categories: First the App calculates B4B, a tool that drives the user to choose those foods that best meet a health weight management plan while addressing dietary restrictions (if any). This is the “looking forward” feature of the App. Second, the App “looks backward” in time each day and documents the user's actual performance in addressing the dietary objectives and special needs incorporated into the weight management plan. This motivates a user to correct bad habits, and reinforces good habits, the results of which are displayed in the historical records being maintained on the computer.

### Calculations for the Base B4B One Number Rating

### Example B4B Calculations

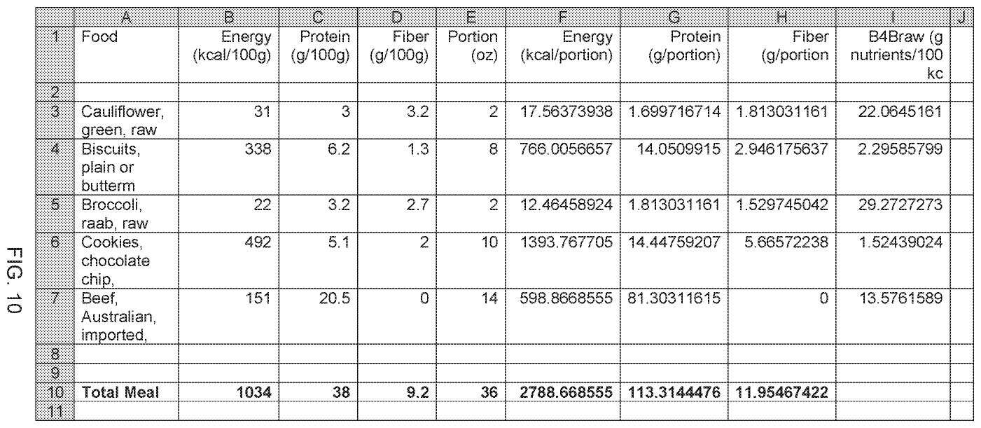

FIGS. 9-13 show a spreadsheet that illustrates values for calculation of the Base B4B One Number rating calculated for an individual food item, as well as for 5 foods making up an example meal.

The base component of the B4B_{base} equation is:

B4B_{base}=100*[g protein+(1.2*g fiber)]/kcal

Note that when evaluating a single food, the portion size doesn't matter, B4B will be the same regardless. It's just how many grams (g) of protein, fiber, and the number of kcals in that portion.

For equations (2.6)-(2.17), the values of the factors, F_{Ch}, F_{Lipids}, F_{EDen}, F_{FD}, and F_{Na} are given in the following equations (2.1-2.5) (all factors are unitless)

F_{Ch}=Cholesterol Concentration in Food (mg Cholesterol/100 g food)·(0.05) Equation (2.1)

F_{Lipids}=Fat Concentration in Food (g Fat/100 g food)·(0.10) Equation (2.2)

F_{EDen}=(Calories/100 g food)·(0.01) tm Equation (2.3)

F_{FD}=Fiber Density of Food (Fiber g/100 g food)·(0.25) Equation (2.4)

F_{Na}=Sodium Content of Food (Sodium mg/100 g food)·(0.02) Equation (2.5)

Taking the example of Biscuits (line 4 of FIG. 9): 100 g (i.e., 3.53 ounces) of Biscuits have 338 kcal, 6.2 g of protein, and 1.3 g of fiber (Columns B, C, D are FDA values of each for 100 g of Biscuits). Base B4B is as follows:

B4B_{base}=100*[6.2 g+(1.2*1.3 g)]/338 kcal

Which works out to a B4Bbase number of 2.3.

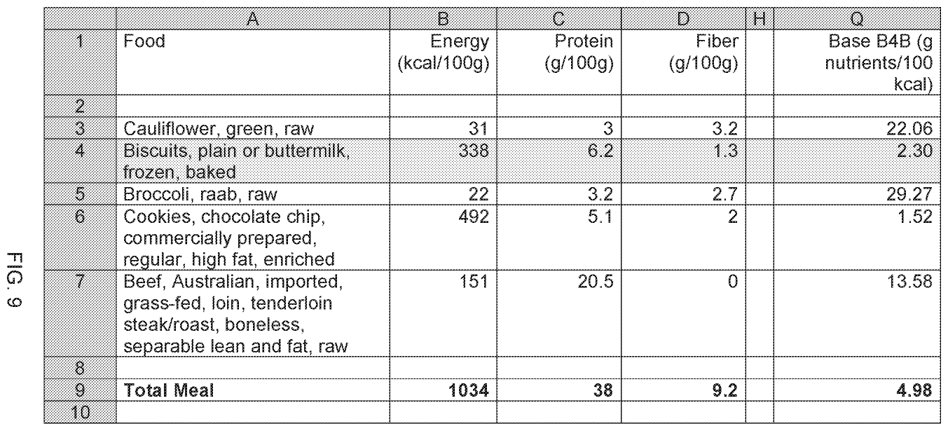

That's for 100 g. If we eat 8 oz. (227 g) of Biscuits, the portion-weighted values (FIG. 10, columns F, G, H) are: 766 kcal, 14 g protein, and 3 g fiber. To obtain these portion weighted values, multiply the 100 g values by portion size (in ounces) and divide by 3.53 (the number of ounces in 100 g). Equation as is follows:

Portion-weighted value (of anything)=100 g value*portion size (oz)/3.53

Once we have the portion-weighted values (columns F, G, H), B4B_{base} works out to:

B4B_{base}=100*[14 g+(1.2*3 g)]/766 kcal

which equals 2,3 again.

### Calculating the B4B Rating Number for a Meal

When considering two or more foods to make a meal, the portion sizes matter (there is a difference between 1000 grams of broccoli and 1 gram of butter vs the inverse). Computing B4B_{base} for a meal is a simple matter of computing the total g of protein and fiber as well as the total kcal for that meal and using the same equation as used in calculating the-B4B_{base} value for a single food item.

FIG. 9. shows nutrient values and B4B calculations for a meal consisting of five foods in varying portion sizes. The total number of ounces in the meal is 36—our hypothetical eater went heavy on the biscuits, cookies, and beef.

The standard 100 g values for protein, fiber, and kcals (B, C, D) have been transformed to portion-weighted values (F, G, H). All told, our eater had 2788 kcals, 113 g of protein, and 12 g of fiber. The B4B_{base} for the meal works out to:

B4B_{base}=100* [113 g+(1.2*12 g)]2788 kcal

Which equals 4.57. Why so low? All of those biscuits and cookies, which have a terrible B4B.

### Using the Correction Factors for Specific Individual Requirements

An important feature of the present invention is that the B4B rating number is factored to meet the different specific health and weight management objectives of individual users. The App employs 12 distinct B4B formulas. The B4B formula to be used will be determined by which one category of objectives and special health needs the user selects. See Table 3.

The App employs 12 distinct B4B formulas. The B4B formula to be used will be determined by which one category of objectives and special health needs the user selects. The 12 categories are shown in Table 3.

TABLE 3

| | OBJECTIVE & SPECIAL | USE |
|---|---|---|
| CATEGORY | HEALTH NEEDS | EQUATION |
| 1 | Maintain weight, no dietary restrictions | (2.6) |
| 2 | Maintain weight, low sodium restriction | (2.7) |
| 3 | Maintain weight, low cholesterol, | (2.8) |
| | low fat restriction | |
| 4 | Maintain weight, low sodium, low | (2.9) |
| | cholesterol, low fat restriction | |
| 5 | Lose weight, no dietary restrictions | (2.10) |
| 6 | Lose weight, low sodium restriction | (2.11) |
| 7 | Lose weight, low cholesterol, | (2.12) |
| | low fat restriction | |
| 8 | Lose weight, low sodium, low | |
| | cholesterol, low fat restriction | (2.13) |
| 9 | Gain weight, no dietary restrictions | (2.14) |
| 10 | Gain weight, low sodium restriction | (2.15) |
| 11 | Gain weight, low cholesterol, | (2.16) |
| | low fat restriction | |
| 12 | Gain weight, low sodium, low | (2.17) |
| | cholesterol low fat restriction | |

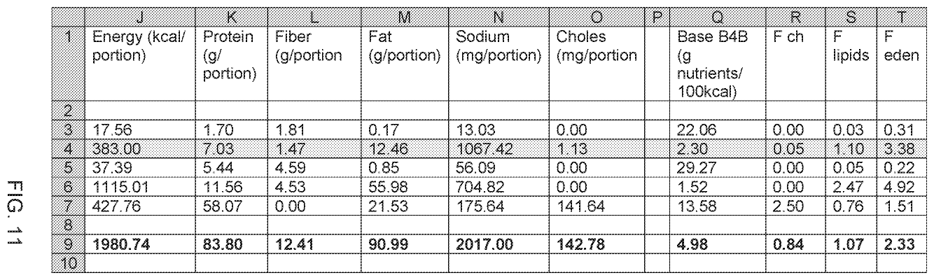

For an individual food, the correction factors work as follows. (We'll use Biscuits, line 4, as our example.) Referring to FIGS. 12 and 13;

F_{Ch} (Column N)=mg cholesterol/100 g*0.05

Biscuits have 1 mg/100 g of cholesterol (see cell G4), thus F_{Ch}=1*0.05=0.05 (as shown in cell N3)

F_{Lipids}(Column O)=g fat/100 g *0.1

Biscuits have 11 g/100 g of fat (cell E4), thus F_{Lipids}=11*0.1=1.1 (as shown in cell O4)

F_{EDen} (Column P)=kcal/100 g*0.01

Biscuits have 338 kcal in 100 g, thus F_{EDen}=338*0.01=3.38 (as shown in cell P4)

F_{FD} (Column Q)=g fiber/100 g*0.25

Biscuits have 1.3 g/100 g of fiber, thus F_{FD}=1.3*0.25=0.325 (as shown in cell Q4)

F_{Na} (Column R)=mg sodium/100 g*0.02

Biscuits have 942 mg of sodium, thus F_{Na}=942*0.02=18.84 (as shown in cell R4)

Note that the factors are not affected by portion size, they are based on numbers computed from 100 g portions.

Nov that we have the correction-factors, we can compute the 12 B4B values for individual foods.

All 12 values start with the Base B4B and then add or subtract various correction factors.

As covered in Part 1, Base B4B is: B4B_{base}=100*[g protein+(1.2* g fiber)]/kcal

### Maintain Weight

B4B₁=Base+F_{FD} Equation (2.6)

Using Biscuits again as our example, B4B₁=2.3+0.325=2.62

B4B₂=Base+F_{FD}−F_{Na} Equation (2.7)

Using Biscuits again as our example, B4B₂=2.3+0.325−18.84=−16.22

B4B₃×=Base+F_{FD}−F_{Ch}−F_{Lipids} Equation (2.8)

Using Biscuits again as our example, B4B₃=2.3+0.325−0.05−1.1=1.47

B4B₄=Base+F_{FD}−F_{Na}−F_{Ch}−F_{Lipids} Equation (2.9)

Using Biscuits again as our example, B4B₄=2.3+0.325−18.84−0.05−1.1 =−17.37

### Lose Weight

B4B₅=Base+F_{FD}−F_{EDen} Equation (2.10)

Using Biscuits again as our example, B4B₅=2.3+0.325−3.38=−0.755

B4B₆=Base+F−F−F_{EDen} Equation (2.11)

Using Biscuits again as our example, B4B₆=2.3+325−18.84−3.38=−19.595

B4B₇=Base+F_{FD}−F_{Ch}−F_{Lipids}−F_{EDen} Equation (2.12)

Using Biscuits again as our example, B4B₇=2.3+0.325−0.05−1.1−3.38=−1.91

B4B₈=Base+F_{FD}−F_{Na}−F_{Ch}−F_{Lipids}−F_{EDen} Equation (2.13)

Using Biscuits again as our example, B4B₈=2.3+0.325−18.84−0.05−1.1−3.38=−20.75

### Gain Weight

B4B₉=Base+F_{FD}F_{EDen} Equation (2.14)

Using Biscuits again as our example, B4B₉=2.3+0.325+3.38=6.001

B4B₁₀=Base+F_{FD}−F_{Na}+F_{EDen} Equation (2.15

Using Biscuits again as our example, B4B₁₀=2.3+0.325−18.84+3.38=−12..839

B4B₁₁=Base+F_{FD}−F_{Ch}−F_{Lipids}+F_{EDen} Equation (2.16)

Using Biscuits again as our example, B4B₁₁=2.3+0.325−0.05−1.1+3.38=4.851

B4B₁₂=Base+F_{FD}−F_{Na}−F_{Ch}−F_{Lipids}+F_{EDen} Equation (2.17)

Using Biscuits again as our example, B4B₁₂=2.3+0.325−18.84−0.05−1.1+3.38=−13.985

### Notes On the B4B Calculations

All B4B1-12 values are limited to 0-20, so anything less than 0=0 and anything greater than 20=20. Should portion size=0, then you get a division by zero error. In that case, set B4B=0.

### Information Provided in the Computer App

FIGS. 14 through 39 illustrate the teachings of the present invention implemented with the App of the present invention installed on a smart phone device. FIGS. 14 through 39 are screenshots taken from a smart phone device.

FIG. 14 illustrates the HOME SCREEN of the App. The SCREEN is scrollable to enable a single page display of some of the most important considerations in the user's weight management plan. The B4B indicator shows the quality of the food already consumed by the App user since midnight. The fat indicator shows the calculated change in the user's weight, illustrated in TUF units, during the same monitored period.

The 3rd important element shown at the very top of the HOME SCREEN is the total fiber indicator that illustrates the user's current status relative to the fiber consumption goal set forth in the weight management plan.

The next information in order of importance is the net calories display showing the difference between the calories consumed and the calories burned since midnight up to the present time.

The following items on the first page display show summaries for the user's physical activities, the calculated calories burned as a result of the activities the users RMR, and the time that the user has been at different levels of heart rate, including the time the user is in the fat burn zone.

The final item on the HOME SCREEN is the log of water consumption during the day. The display shows the user's status relative to the water consumption goal included as a part of the user's weight management program. Encouraging comments are used to prompt the user to consume more water.

FIGS. 14 through 17 illustrate a feature of the present invention that enables a user to test a proposed food against the important nutrient and health parameters being monitored in the weight management plan. FIG. 14 illustrates a B4B value of 8 and a fat value of −3 tablespoons.

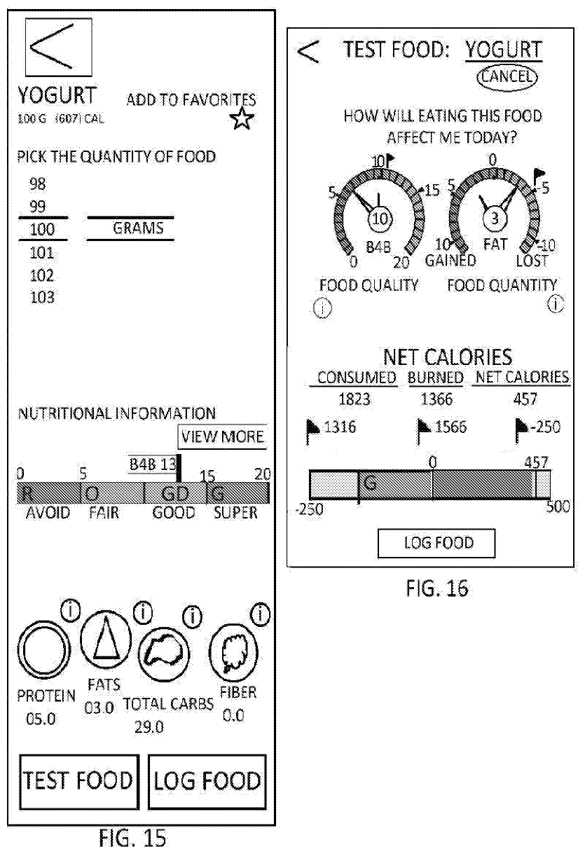

The user proposes eating a serving of yogurt and selects that item from the database of the App which produces the display illustrated in FIG. 15. The bottom of Fla 15 gives the user two options, to test the food or to log it. Selecting the test food option will present the screen shown in FIG. 16. The illustration of FIG. 16 displays that consuming the serving of yogurt would increase the B4B quality of the food consumed thus far from an 8 to a 10, however the user will have moved from a loss of 3 tablespoons of fat to a gain of 3 tablespoons of fat beyond the user's energy bum. The single serving of yogurt contains approximately 600 Calories, the amount of approximately 6 tablespoons of body fat.

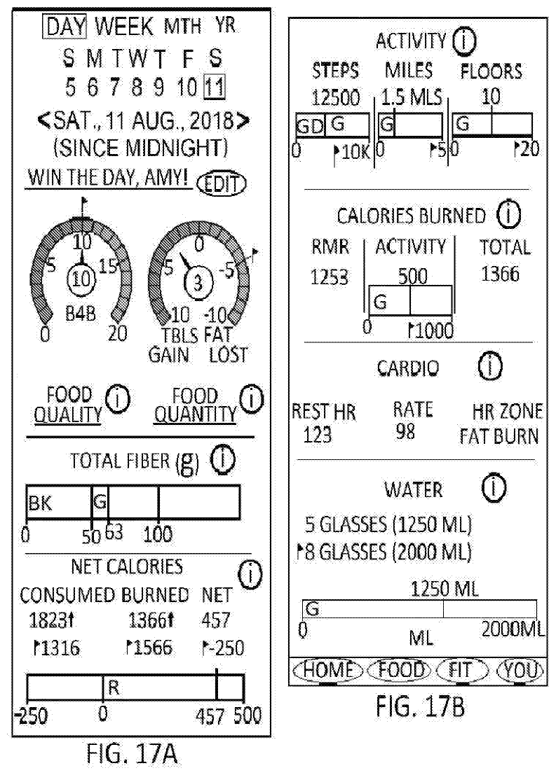

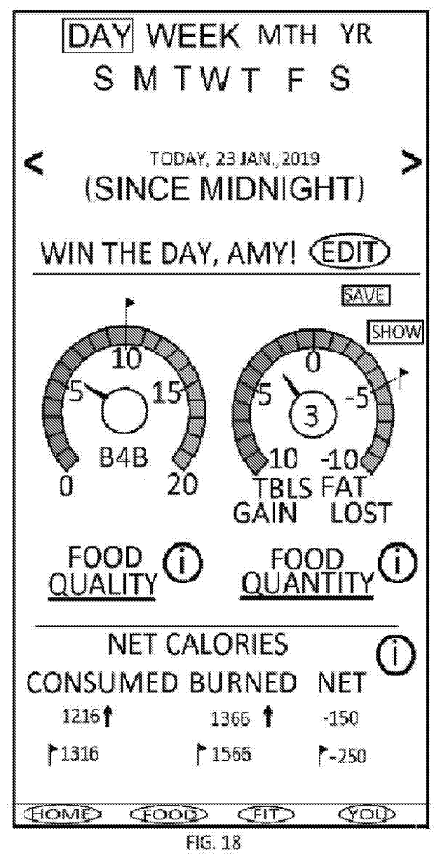

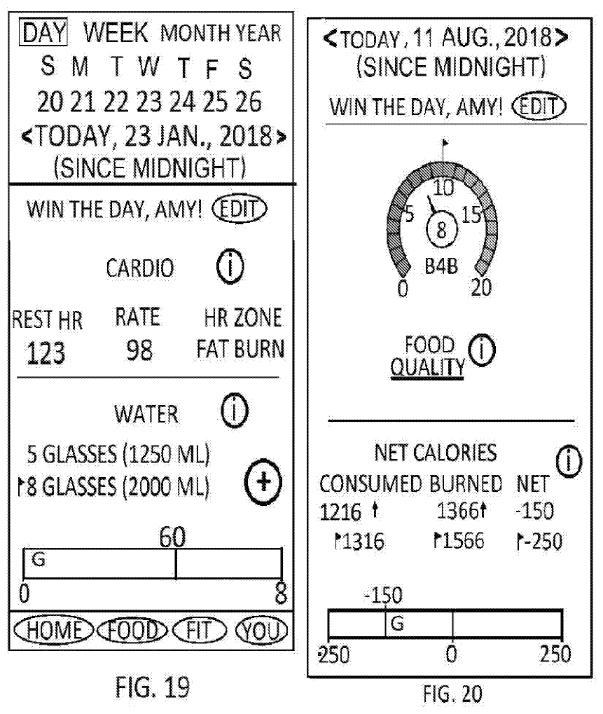

FIGS. 18 and 19 makeup, respectively, the upper and lower halves of the HOME SCREEN. One of the features of the present invention is that the display of the HOME SCREEN may be edited by the user to rearrange the display to present those features most important to the individual toward the top of the display. FIG. 20 illustrates one of the possible editing rearrangements.

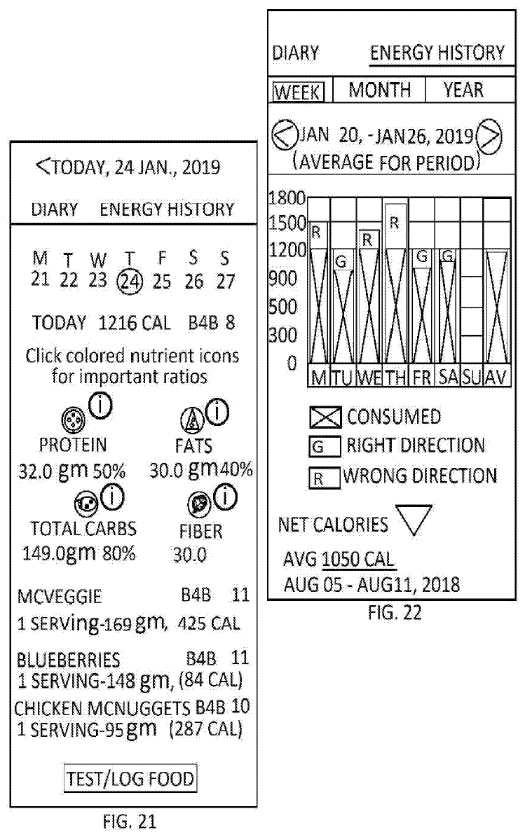

When the user selects the “FOOD” option at the bottom of the FIG. 14, the image shown in FIG. 21 replaces that shown in FIG. 14. FIG. 21 displays information summarizing the current status of Calorie consumption, B4B value, attainment of desired ratios of important nutrients, food eaten and logged with the corresponding B4B number for each food item. The TEST/LOG FOOD option is available at this point in the program to permit evaluation of specific proposed food consumption.

The display of FIG. 22 appears when the “ENERGY HISTORY” heading is selected. Over consumption and under consumption levels are indicated by coloration of the tops of the bar graphs. This display, as with the others in the

APP are intended to present vivid, immediately understandable and appreciated information about the user status in attaining the objectives and goals of the selected weight management program.

As with other of the monitored parameters in the App, where appropriate, a historical record is maintained on a daily, weekly, monthly and in some cases a yearly basis.

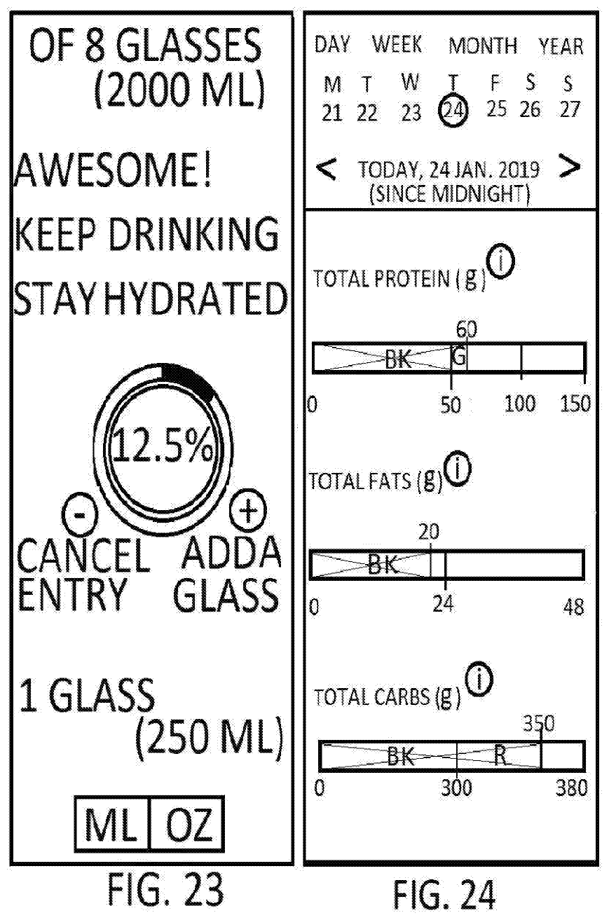

FIG. 23 illustrates the status of the user's logged water consumption. Incentivizing comments are provided to encourage sufficient water consumption.

FIG. 24 illustrates the basic nutrients being monitored and the status for the day.

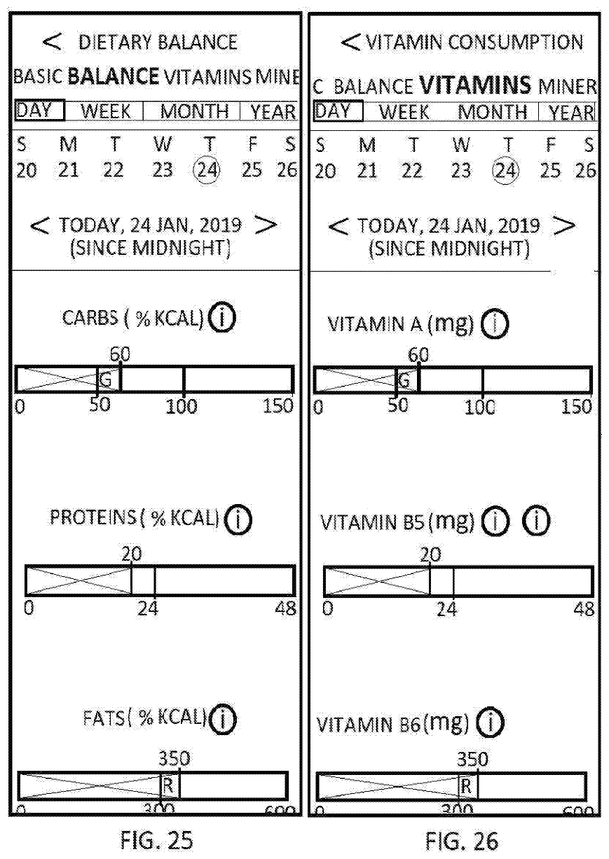

FIG. 25 illustrates the nutrients being monitored to evaluate the dietary balance of the foods being consumed.

FIG. 26 illustrates the monitoring of vitamin consumption;

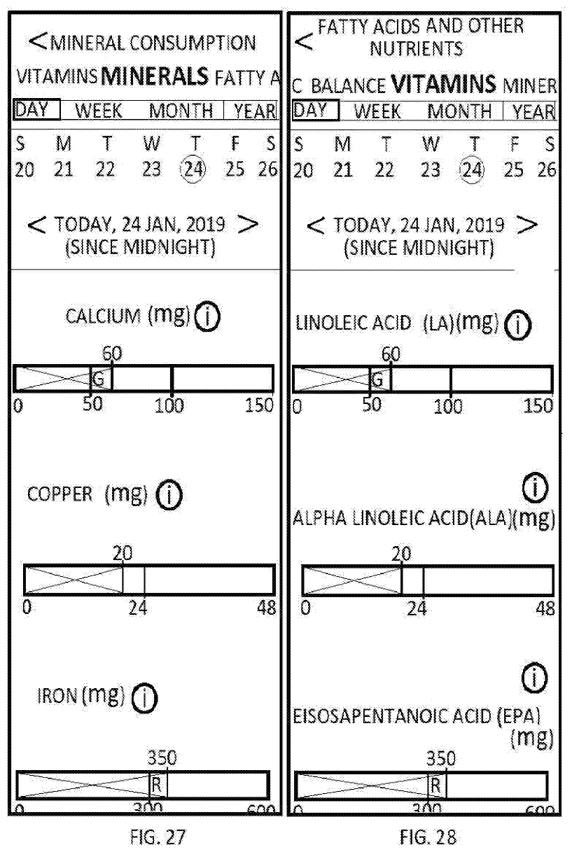

FIG. 27 illustrates the monitoring of mineral consumption;

FIG. 28 illustrates monitoring of FATTY ACIDS/and OTHERS;

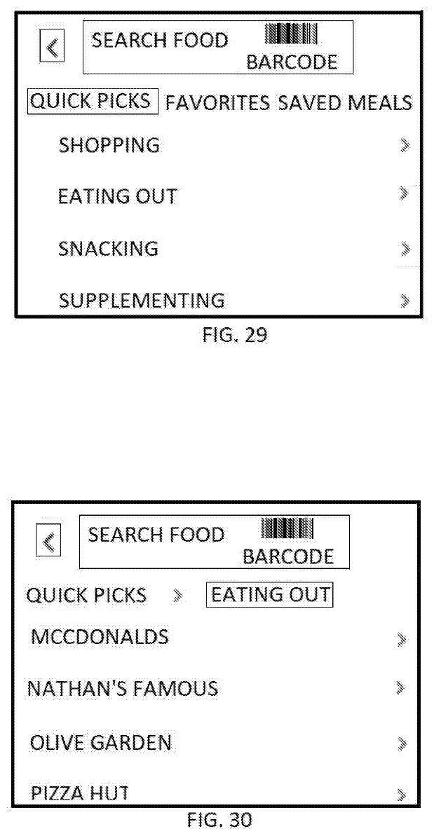

FIG. 29 illustrates 4 categories of food sources to assist the user in selecting and evaluating food to be consumed;

FIG. 30 illustrates a FAVORITES listing of foods that is developed as a user implements the tool of the invention;

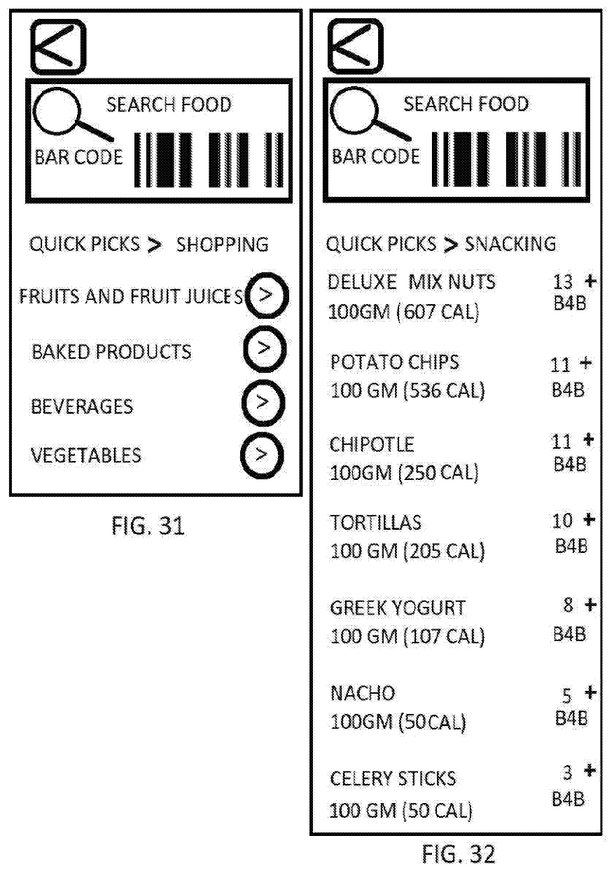

FIG. 31 illustrates recently eaten foods;

FIG. 32 illustrates foods categorized under the snacking heading;

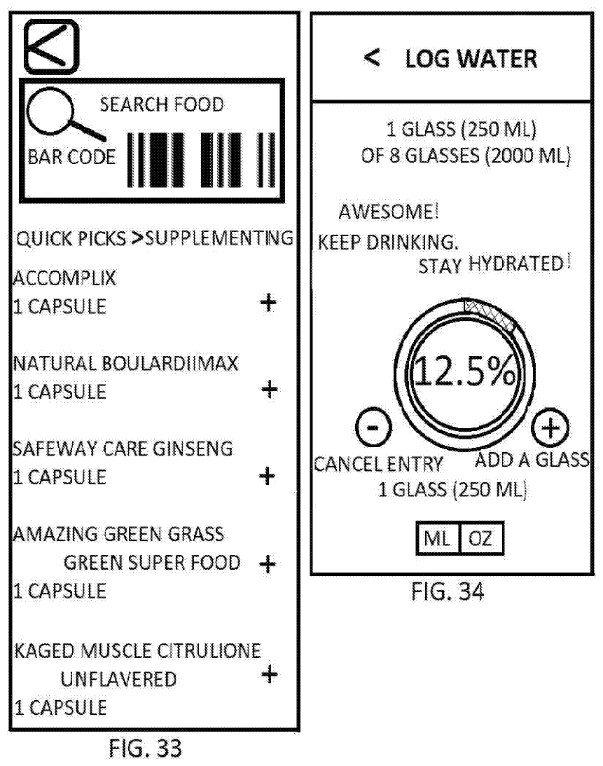

FIG. 33 illustrates a data listing of nutritional supplements;

FIG. 34 illustrates the user's status consuming water. Water consumption status is presented in multiple places in the APP to encourage the user to consume sufficient quantities of water;

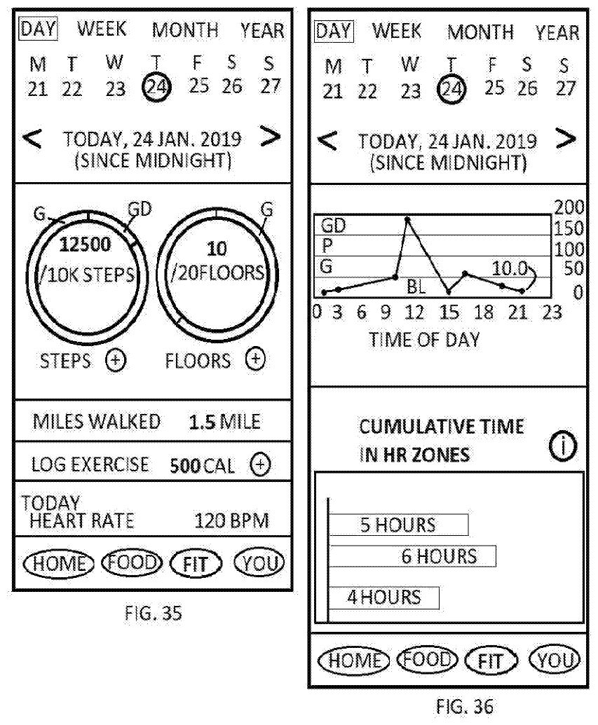

FIG. 35 is the activity record for the user showing number of Steps taken, number of stair Flights climbed, Miles walked and other exercise logged during the monitored period since midnight. Heart rate and time in the heart rate zones are also monitored and recorded.

FIG. 36 is the bottom half of FIG. 35 illustrating the heart rate record.

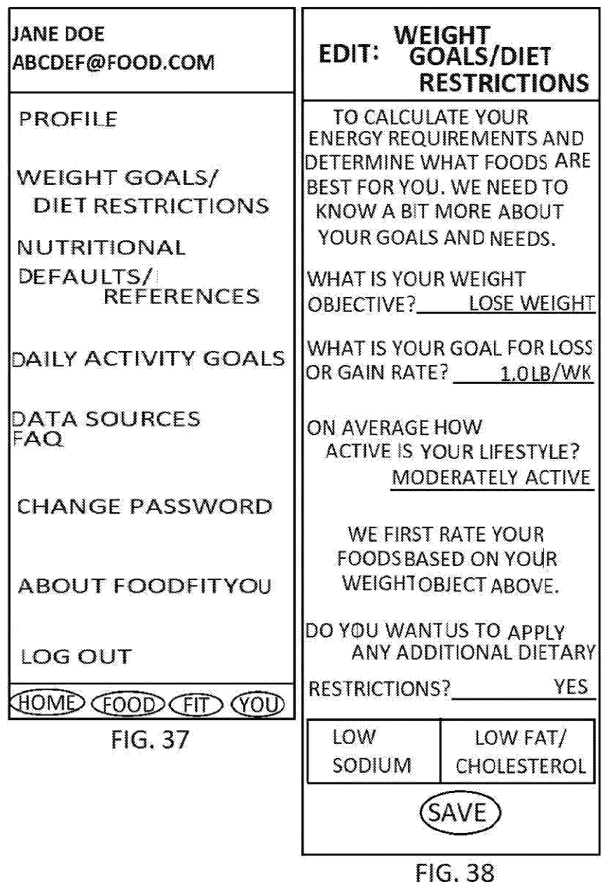

FIG. 37 provides the user identity and physical information used in formulating and establishing the weight management program for the individual.

FIG. 38 provide specific information regarding the individual's objectives and personal nutritional and health interests.

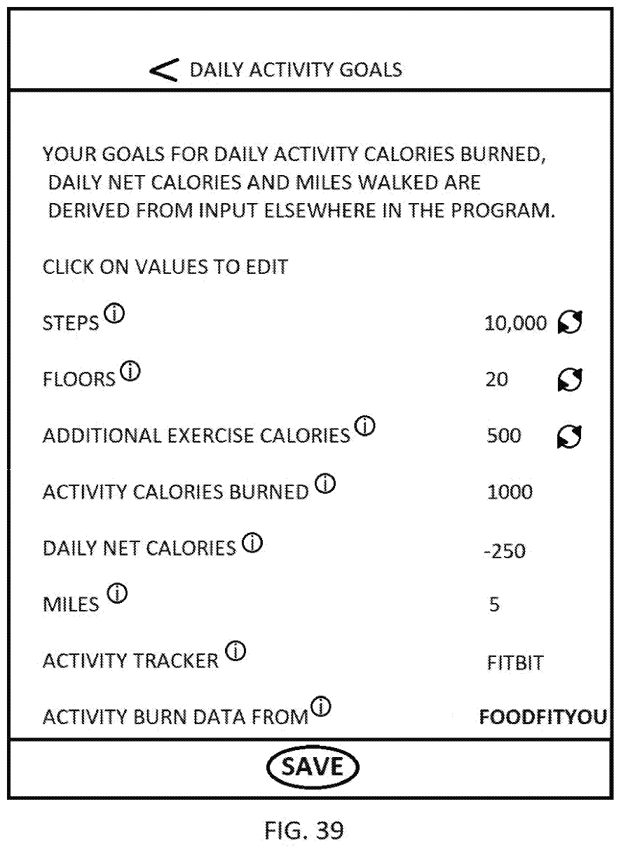

FIG. 39 specifies the user's daily activity goals.

Although specific embodiments of the invention have been described herein in some detail, this has been done solely for the purposes of explaining the various aspects of the invention, and is not intended to limit the scope of the invention as defined in the claims which follow. Those skilled in the art will understand that the embodiment shown and described is exemplary, and various other substitutions, alterations and modifications, including but not limited to those design alternatives specifically discussed herein, may be made in the practice of the invention without departing from its scope.

### Calculations and Data

$$FORMULAS FOR {ENGINEER}^{′} S non \text{-} Diet$$

$$Basic B 4 B RATING SYSTEM$$

$$1.$$

$$B 4 {B}_{F} ( g fiber \text{/} 100 Calories ) = \frac{100 * ( g fiber ) ( 100 g food )}{Calories \text{/} 100 g food}$$

$$2.$$

$$B 4 {B}_{F} ( g protein \text{/} 100 Calories ) = \frac{100 * ( g protein ) ( 100 g food )}{Calories \text{/} 100 g food}$$

$$3. B 4 {B}_{TOTAL} = B 4 {B}_{F} + B 4 {B}_{F}$$

$$4.$$

$$MILES WALKED = \frac{HEIGHT ( in . ) * 0.425 * STEPS WALKED}{63 , 360}$$

$$( STEPS WALKED is input from activity tracker )$$

$$5.$$

$$CALORIES BURNED per FLOOR CLIMBED = ( WEIGHT ( lbs . ) * 0.0216 )$$

$$6.$$

$$CALORIES BURNED per MILE WALKED = ( WEIGHT ( lbs . ) * 0.531 ( Cal \text{/} lb ) )$$

$$7.$$

$$CALORIES BURNED per STEP WALKED = \frac{\begin{matrix}( HEIGHT ( in . ) * 0.425 ) ) * \\ ( WEIGHT ( lbs . ) * 0.531 ( Cal \text{/} lb ) )\end{matrix}}{63 , 360}$$

$$8.$$

$$NET CALORIES = ( CALORIES EATEN ) - ( CALORIES BURNED ) during a period$$

$$9.$$

$$RMR \text{/} MINUTE ( Calories burned \text{/} minute = \frac{\begin{matrix}( 10 * WEIGHT ( kg ) ) + \\ ( 6.25 * HEIGHT ( cm ) ) - ( 5 * AGE ( years ) ) + n\end{matrix}}{1440} \\ ( where n is + 5 for males and - 161 for females ) \\ 10. \\ ( CALORIES BURNED ( since midnight ) = ( RMR \text{/} MINUTE ) * ( MINUTES SINCE MIDNIGHT ) ) + ( \begin{matrix}( STEPS SINCE MIDNIGHT ) * \\ [ \frac{\begin{matrix}( HEIGHT ( in . ) * 0.425 ) ) * \\ ( WEIGHT ( lbs . ) * 0.531 ( Cal \text{/} lb ) )\end{matrix}}{( 63 , 360 )} ]\end{matrix} ) + ( ( WEIGHT ( lbs . ) * 0.0216 ) * FLOORS CLIMBED SINCE MIDNIGHT ) ) + ( OTHER CALORIES SINCE MIDNIGHT ) \\ 11. FAT GAINED ( + ) OR LOST ( - ) = \frac{NET CALORIES}{105.2} \\ ( DURING A PERIOD \text{-} IF NET CALORIES IS \\ ( - ) FAT IS LOST AND VICE VERSA ) \\ 12. \\ AVERAGE B 4 B = \frac{100 * [ \begin{matrix}{\sum }_{THIS PERIOD} PROTEIN EATEN ( g ) + \\ {\sum }_{THIS PERIOD} FIBER EATEN ( g )\end{matrix} ]}{{\sum }_{THIS PERIOD} CALORIES EATEN ( g )} \\ \\ ( PERIOD MAY BE SINCE MIDNIGHT OR MONTHLY \\ AVG FOR MONTHLY SCREENS ) \\ 13. \\ PROTIEN = {\sum }_{THIS PERIOD} PROTEIN EATEN ( g ) ( PERIOD MAY BE SINCE MIDNIGHT OR - MONTHLY AVG \text{/} DAY FOR MONTHLY SCREENS ) \\ 14. FIBER = {\sum }_{THIS PERIOD} FIBER EATEN ( g ) \\ ( PERIOD MAY BE SINCE MIDNIGHT OR MONTHLY \\ SCREENS ) \\ 15. CHOLESTEROL = {\sum }_{THIS PERIOD} CHOLESTEROL EATEN ( g ) \\ ( PERIOD MAY BE SINCE MIDNIGHT OR MONTHLY \\ SCREENS ) \\ NOTE : EQUATION 16 APPLIES FOR VITAMINS , \\ MINERALS , FATTY ACIDS AND SPECIALTY \\ NUTRIENTS . \\ 16. \\ COMPONENT = {\sum }_{THIS PERIOD} COMPONENT EATEN ( g ) ( PERIOD MAY BE SINCE MIDNIGHT OR MONTHLY AVG \text{/} DAY FOR MONTHLY SCREENS , HOWEVER , ONCE USER REACHES THE DAILY GOAL FOR A COMPONENT IN A GROUP ( IE \text{:} VITAMIN C IN THE VITAMIN GROUP ) , FURTHER CONSUMPTION OF VITAMIN C DOES NOT ADD TO THE TOTAL FOR VITAMIN GROUP . )$$

### FORMULAS FOR B4B APP TOOL

$$\begin{matrix} Equations ( 1.1 ) , ( 1.2 ) and ( 1.3 ) are \\ algorithms for the unfactored B 4 B rating system \text{:} \\ & \\ 1. \\ B 4 {B}_{F} ( g fiber \text{/} 100 Calories ) = \frac{100 * ( g fiber ) / ( 100 g food )}{Calories \text{/} 100 g food} & equation ( 1.1 ) \\ 2. \\ B 4 {B}_{F} ( g protein \text{/} 100 Calories ) = \frac{100 * ( g protein ) / ( 100 g food )}{Calories \text{/} 100 g food} & equation ( 1.2 ) \\ 3. B 4 {B}_{TOTAL} = B 4 {B}_{F} + B 4 {B}_{F} & equation ( 1.3 )\end{matrix}$$

2.1) Calculated Quantities

The app will perform and either use internally or display the results of calculations that employ the App data input. These calculations fall into two broad categories: First the app B4B, a tool that drives the aver to choose those foods that best meet his health objectives and dieting restrictions (if any). This is the “looking forward” feature of the app. Second, the app “looks backward” in time each day and documents the user's actual performance against his dietary objectives and special needs. This motivates a user to correct bad habits, and reinforces good habits, the results of which are displayed in his daily and historical records.

2.1 B8B Food Ranking Tool

The F4B One Number App uses 1.2 distinct B4B formulas. The B4B formula is determined by which one category of objectives and special health needs the user selects. The 12 categories are:

| | OBJECTIVE & SPECIAL | USE |
|---|---|---|
| CATEGORY | HEALTH NEEDS | EQUATION |
| 1 | Maintain weight, no dietary restrictions | (2.6) |
| 2 | Maintain weight, low sodium restriction | (2.7) |
| 3 | Maintain weight, low cholesterol, | (2.8) |
| | low fat restriction | |
| 4 | Maintain weight, low sodium, low | (2.9) |
| | cholesterol, low fat restriction | |
| 5 | Lose weight, no dietary restrictions | (2.10) |
| 6 | Lose weight, low sodium restriction | (2.11) |
| 7 | Lose weight, low cholesterol, | (2.12) |
| | low fat restriction | |
| 8 | Lose weight, low sodium, low | (2.13) |
| | cholesterol, low fat restriction | |
| 9 | Gain weight, no dietary restrictions | (2.14) |
| 10 | Gain weight, low sodium restriction | (2.15) |
| 11 | Gain weight, low cholesterol, | (2.16) |
| | low fat restriction | |
| 12 | Gain weight, low sodium, low | (2.17) |
| | cholesterol, low fat restriction | |

B4B Formulas

For equations 2.6-2.17, the values of the factors, F_{Ch}, F_{Lipids}, F_{EDen}, F_{FD}, and F_{Na} ate given in the following formulas 2.1-2.5. (All factors are unitless):

F_{Ch}=Cholesterol Concentration in Food (mg Cholesterol/100 g food)·(0.05) eq. (2.1)

F_{Lipids}=Fat Concentration in Food (g Fat/100 g food)·(0.10) equation (2.2)

F_{EDen}=(Calories/1.00 g food)·(0.01) equation (2.3)

F_{FD}=Fiber Density of Food (Fiber g100 g food(·0.25) equation (2.4)

F_{Na}=Sodium Content of Food (Sodium mg/100 g food)·(0.02) equation (2.5)

The 12 formulas for B4B referenced in paragraph 2.1 are:

$$\begin{matrix} Category 1 , Maintain weight , no dietary restrictions ( B 4 {B}_{1} ) & \\ B 4 {B}_{1} = \frac{g protein + ( 1.2 \cdot g fiber )}{100 Calories} + {F}_{FD} & equation ( 2.6 ) \\ Category 2 , Maintain weight , low sodium restriction ( B 4 {B}_{2} ) & \\ B 4 {B}_{2} = \frac{g protein + ( 1.2 \cdot g fiber )}{100 Calories} + {F}_{FD} - {F}_{Na} & equation ( 2.7 ) \\ Category 3 , Maintain weight , low cholesterol restriction ( B 4 {B}_{3} ) & \\ B 4 {B}_{3} = \frac{g protein + ( 1.2 \cdot g fiber )}{100 Calories} + {F}_{FD} - {F}_{Ch} - {F}_{Lipids} & equation ( 2.8 ) \\ Category 4 , Maintain weight , \\ low sodium , low fat , \\ low cholesterol restriction ( B 4 {B}_{4} ) & \\ B 4 {B}_{4} = \frac{g protein + ( 1.2 \cdot g fiber )}{100 Calories} + {F}_{FD} - {F}_{Ch} - {F}_{Lipids} - {F}_{Na} & equation ( 2.9 ) \\ Category 5 \text{:} Lose weight , \\ no dietary restrictions ( B 4 {B}_{5} ) & \\ B 4 {B}_{5} = \frac{g protein + ( 1.2 \cdot g fiber )}{100 Calories} + {F}_{FD} - {F}_{EDen} & equation ( 2.10 ) \\ Category 6 \text{:} Lose weight , \\ low sodium restrictions ( B 4 {B}_{6} ) & \\ B 4 {B}_{6} = \frac{g protein + ( 1.2 \cdot g fiber )}{100 Calories} + {F}_{FD} - {F}_{EDen} - {F}_{Na} & equation ( 2.11 ) \\ Category 7 \text{:} Lose weight , low fat , \\ low cholesterol restrictions ( B 4 {B}_{7} ) & \\ B 4 {B}_{7} = \frac{g protein + ( 1.2 \cdot g fiber )}{100 Calories} + {F}_{FD} - {F}_{EDen} - {F}_{Ch} - {F}_{Lipids} & equation ( 2.12 ) \\ Category 8 \text{:} Lose weight , \\ low sodium , low fat , \\ low cholesterol restrictions ( B 4 {B}_{8} ) & \\ B 4 {B}_{8} = \frac{g protein + ( 1.2 \cdot g fiber )}{100 Calories} + {F}_{FD} - {F}_{Eden} - {F}_{Ch} - {F}_{Na} - {F}_{Lipids} & equation ( 2.13 ) \\ Category 9 \text{:} Gain weight , \\ no dietary restrictions ( B 4 {B}_{9} ) & \\ B 4 {B}_{9} = \frac{g protein + ( 1.2 \cdot g fiber )}{100 Calories} + {F}_{FD} + {F}_{EDen} & equation ( 2.14 ) \\ Category 10 \text{:} Gain weight , \\ low sodium restrictions ( B 4 {B}_{10} ) & \\ B 4 {B}_{10} = \frac{g protein + ( 1.2 \cdot g fiber )}{100 Calories} + {F}_{FD} + {F}_{EDen} - {F}_{Na} & equation ( 2.15 ) \\ Category 11 \text{:} Gain weight , \\ low cholesterol restriction ( B 4 {B}_{11} ) & \\ B 4 {B}_{11} = \frac{g protein + ( 1.2 \cdot g fiber )}{100 Calories} + {F}_{FD} + {F}_{EDen} - {F}_{Ch} - {F}_{Lipids} & equation ( 2.16 ) \\ Category 12 \text{:} Gain weight , \\ low sodium , low fat , \\ low cholesterol restriction ( B 4 {B}_{12} ) & \\ B 4 {B}_{12} = \frac{g protein + ( 1.2 \cdot g fiber )}{100 Calories} + {F}_{FD} + {F}_{EDen} - {F}_{Na} - {F}_{Ch} - {F}_{Lipids} & equation ( 2.17 )\end{matrix}$$

2.2 Calculated Dietary Balance Ratios

These calculations “look backward” in time each day and document the user's actual performance against his dietary objectives, needs and Recommended Daily Allowances of the 11 ratios that track the user's dietary balance. These ratios are;

BALANCE

| | |
|---|---|
| 11 | FOOD QUALITY INDEX (B4B) |
| I2 | FIBER INDEX (FIBER/TOTAL CARBS) (g/g) |
| I3 | FAT SOURCE INDEX (PLANT FATS/TOTAL FATS) (g/g) |
| I4 | PROTEIN SOURCE INDEX (PLANT PROT/TOTAL PROT) (g/g) |
| I5 | FATTY ACID INDEX (DHA/EPA) (mg/mg) |
| I6 | OMEGA INDEX (OMEGA-6/OMEGA-3) (mg/mg) |
| R1 | CARBS (% Kcal) |
| R2 | PROTEINS (% Kcal) |
| R3 | FATS (% Kcal) |
| R4 | SATURATED FATS (% kCal) |
| R5 | ADDED SUGARS (% kCAL) |

2.2 Dietary Balance Formulas

The ratio formulas referenced in paragraphs 1.1 and 2.2 are given below:

$$\begin{matrix} R 1 = Energy Density ( EDen ) = Calories \text{/} 100 g food & equation ( 2.18 ) \\ R 2 = Fiber Density ( FD ) = Fiber g \text{/} 100 g food & equation ( 2.19 ) \\ R 3 = \% carbs = \frac{\sum Total Calories eaten as Carbs}{\sum Total Calories Eaten} \\ in a day or part thereof & equation ( 2.20 ) \\ R 4 = \% protein = \frac{\sum Total Calories eaten as protein}{\sum Total Calories Eaten} \\ in a day or part thereof & equation ( 2.21 ) \\ R 5 = \% fat = \frac{\sum Total Calories eaten as fat}{\sum Total Calories Eaten} \\ in a day or part thereof & equation ( 2.22 ) \\ R 6 = \% added sugars = \frac{\sum Total Calories eaten as added sugar}{\sum Total Calories Eaten} \\ in a day or part & equation ( 2.23 ) \\ R 7 = fiber \text{/} total carbs = \frac{\sum Total fiber eaten g}{\sum Total Carbs Eaten g} \\ in a day or part thereof & equation ( 2.24 ) \\ R 8 = \% veg fat = \frac{\sum Vegetable fat eaten g}{\sum Total fat eaten g} \\ in a day or part thereof & equation ( 2.25 ) \\ R 9 = \% veg protein = \frac{\sum Vegetable protein eaten g}{\sum Total protein eaten g} \\ in a day or part thereof & equation ( 2.26 ) \\ R 10 = DHA \text{/} EPA = \frac{\sum DHA eaten g}{\sum EPA eaten g} \\ in a day or part thereof & equation ( 2.27 ) \\ R 2 PROTEINS ( \% Kcal ) & \\ R 3 FATS ( \% Kcal ) & \\ R 4 SATURATED FATS ( \% kCal ) & \\ R 5 ADDED SUGARS ( \% kCAL ) & \\ R 11 = Omega \text{-} 6 / Omega \text{-} 3 = \frac{\sum Omega \text{-} 6 eaten mg}{\sum Omega \text{-} 3 eaten mg} \\ in a day or part thereof & equation ( 2.28 )\end{matrix}$$

3.0 Calculating Calories Burned

3.1 Calculate RMR:

BMR=(4.536 w+15.875 h−5.0 a+5 s−161(1s)) Cal/day (equation 3.1)

Where:
w=user's weight in pounds
h=user's height in inches
a−use's age in years
s=1 for males and 0 for females

3.2 Calculate RAW per minute:

A feature of the app is to give the user a minute-to-minute updated count of calories he is burning. So RMR will also he needed in Calories/minute in addition to its standard unit of Calories/day.

$$RMR \text{/} min = \frac{RMR ( Cal \text{/} day )}{1440 ( min \text{/} day )} ( Cal \text{/} min ) \qquad equation ( 3.2 )$$

3.3 Determine Calories burned by Activity (AC):

The app will continuously calculate (or acquire) and display Activity Calories (AC) during the day. There are three methods. User may choose the one he prefers. The default is Method 1.

Method 1. The app will internally calculate AC using equation 3.3. This is the preferred method. An activity tracker measuring steps is required. If the tracker also measures elevation (floors), Unit data will be used, but is not required. Steps and elevation will be accessed from HealthKit, Google Fit, or Fitbit's data center, depending on phone type or whether the tracker is a Fitbit product.

Method 2. The app will internally calculate AC using equation 3.7. Method 2 is for users who choose not to wear an activity tracker.

Method 3. Some activity trackers may estimate and load AC into Healthkit. If user chooses method 3, the app will not calculate AC, but will simply acquire AC from Health kit and use it wherever AC is called for.

33.1 Method 1:

$$\begin{matrix} A C ( Cal ) = CS ( Cal ) + CF ( Cal ) + CE ( cal ) \\ Where \text{:} & equation ( 3.3 ) \\ CS = ( No . of steps ) * \frac{\begin{matrix}( HEIGHT ( in . ) * ( 0.425 ) ) * \\ ( WEIGHT ( lbs . ) * ( 0.531 ) \end{matrix}}{63 , 360} & equation ( 3.4 ) \\ CF = ( No . of floors ) * ( WEIGHT ( lbs . ) * ( 0.0216 ) & equation ( 3.5 ) \\ CE = RMR \text{/} min * ( minutes spent exercising ) * ( Exercise METs ) & equation ( 3.6 )\end{matrix}$$

where:
Exercise METs=Factor from Table 1 for user-selected exercise

3.3.2 Method 2:

If user selects method 2, he will be asked to describe his lifestyle as either “Not Active,” “Active” or “Very Active.” The app will use the following equation:

AC=RMR/min*(Cumulative minutes since midnight)·F (equation 3.7)

Where:
F=0.15 if user selects lifestyle “Not Active” (default selection)
F=0.26 if user selects lifestyle “Active”
F=0.37 if user selects lifestyle “Very Active”

3.33 Method 3:

The app will acquire AC from Health Kit.

3.4 Daily Total Cumulative Burn (TCB):

In order to give the user a continuous states of his Total Cumulative Burn (TCB) the app will continuously calculate and display this quantity (the word “cumulative” connotes today since midnight):

Total/Cumulative Burn (TCB)=Cumulative (RMR/min)+Cumulative AC (equation 3.8)

Where Cumulative RMR/min=(RMR/min)·(elapsed min from midnight) (equation 3.9)

3.5 Daily Total Burn:

The total Calorie Bum for a day shall be the TCB value on midnight for that day, at which time the TCB will be set to zero and begin accumulating for the following day.

4.0 Displaying Nutrient Minimums, Maximums and Ranges vs. Actual

When displaying actual consumed quantities of a nutrient, if the recommended daily allowance is given as a range, use 4.1. If the RDA is a minimum with no maximum, use 4.2. If the RDA is a maximum with no minimum, use 4.3. The values for maximum and minimum RDA's are taken from user preferences. Default user preferences (which user may change) are established according to the procedure in paragraph 5.0 below.

5.0 Calculating and Entering Default RDA's into User Preferences

A unique feature of the app will be ability to tailor Recommended Daily Nutrient Allowances (RDA's) to the user. RDA's vary depending on gender, age and for females, whether or not the use is pregnant and/or lactating. Basic RDA targets are given in Table 2 and are (for the most part) taken from the 2015-2020 Dietary Guidelines for Americans, published by the US Departments of Health and Agriculture, and from the United States National Academy of Sciences, Institute of Medicine. However, the numbers in table 2 assume a 2000 Calorie/day diet. Corrections for an individual user's estimated individual Caloric intake will be applied to these numbers to assign defaults for individual users. (Users may adjust assigned defaults under advice from medical professionals.)

Find the user's basic RDA from the following Table 2 columns:

| | | USE THESE COLUMN'S |
|---|---|---|
| AGE | GENDER | FOR BASIC RDA's |
| 19-30 | Female | 5 (min) & 6 (max) |
| (NO DIETARY | Male | 7 (min) & 8 (max) |
| RESTRICTION) | | |
| 31-50 | Female | 9 (min) & 10 (max) |
| (NO DIETARY | Male | 11 (min) & 12 (max) |
| RESTRICTION) | | |
| 51-70 | Female | 13 (min) & 14 (max) |
| (NO DIETARY | Male | 15 (min) & 16 (max) |
| RESTRICTION) | | |
| 71+ | Female | 17 (min) & 18 (max) |
| (NO DIETARY | Male | 19 (min) & 20 (max) |
| RESTRICTION) | | |
| Low Sodium | Female | 21 (min) & 22 (max) |
| | Male | 23 (min) & 24 (max) |
| Low Cholesterol, | Female | 25 (min) & 26 (max) |
| fat | Male | 27 (min) & 28 (max) |
| Low Cholesterol, | Female | 29 (min) & 30 (max) |
| fat, Sodium | Male | 31 (min) & 32 (max) |
| Lactating | Female | 33 (min) & 34 (max) |
| Pregnant | Female | 35 (min) & 36 (max) |

5.1 Calculate, User's RMR from Equation 3.1

5.2 Calculate user's estimated Activity burn (AC) from equation 3.7 (F=0.26)

5.3 Calculate user's Adjusted Total Calorie Burn Ratio (ATC Ratio)

ATC Ratio−(RMR+AC)/2000 (equation 5.1)

5.4 If ATC Ratio=1.0

Insert basic values from the appropriate Table 2 columns for minimum and maximum RDA's into preferences.

5.5 If ATC Ratio <1.0

Insert basic values from the appropriate Table 2 column into user's minimum RDA. Multiply user's maximum RDA from Table 2 by ATC Ratio and insert the product for user's maximum.

5.6 If ATC Ratio >1.0:

Insert basic values from the appropriate Table 2 column into user's maximum RDA. Multiply user' minimum RDA from Table 2 by ATC Ratio and insert the product for user's minimum RDA.

1. A method for rating the quality of food in a plurality of foods comprising:
using the quantitative value of each food's energy, protein and fiber content for calculating the energy density of the protein and fiber content of each food;
combining the energy densities of the food to determine a single energy density value for the protein and fiber content of the food;
modifying the single energy density value as a function of the food's fiber content value to provide a basic rating number; and
rating a food with a higher basic rating number as a better quality food than a food with a lower basic rating number.

2. The method of claim 1, wherein the basic rating number is raised or lowered as a function of the quantitative value of one or more specific nutrients in the food in addition to fiber.

3. The method of claim 1, wherein the calculated basic rating number is derived from the food's energy, protein and fiber content, and increased in value as a function of the food's fiber density.

4. The method of claim 2, wherein said one or more specific nutrients is selected from a group of nutrients consisting of lipids, cholesterol, sodium, fiber density and protein density.

5. The method of claim 2, wherein the basic rating number is increased or decreased as a function of the presence of specific nutrients in addition to protein and fiber that are related to specific health concerns of an individual.

6. The method of claim 5, wherein said individual has no specific individual health concerns and wishes to:
a. maintain a healthy weight and the basic rating number for such individual is calculated by the following equation:
$$B 4 {B}_{1} = \frac{g protein + ( 1.2 \cdot g fiber )}{100 Calories} + {F}_{FD} ;$$
or
b. lose weight while maintaining a healthy weight management plan and the basic rating number for such individual is calculated by the following equation:
$$B 4 {B}_{5} = \frac{g protein + ( 1.2 \cdot g fiber )}{100 Calories} + {F}_{FD} - {F}_{EDen} ;$$
or
c. gain weight while maintaining a healthy weight management plan and the basic rating number for such individual is calculated by the following equation:
$$B 4 {B}_{9} = \frac{g protein + ( 1.2 \cdot g fiber )}{100 Calories} + {F}_{FD} + {F}_{EDen} .$$

7. The method of claim 5, wherein the specific health concern of said individual is a low sodium restriction and said individual wishes to:
a. maintain a healthy weight and the basic rating number for such individual is calculated by the following equation:
$$B 4 {B}_{2} = \frac{g protein + ( 1.2 \cdot g fiber )}{100 Calories} + {F}_{FD} - {F}_{Na} ;$$
or
b. lose weight while maintaining a healthy weight management plan and the basic rating number for such individual is calculated by the following equation:
$$B 4 {B}_{6} = \frac{g protein + ( 1.2 \cdot g fiber )}{100 Calories} + {F}_{FD} - {F}_{EDen} - {F}_{Na} ;$$
or
c. gain weight while maintaining a healthy weight management plan and the basic rating number for such individual is calculated by the following equation:
$$B 4 {B}_{10} = \frac{g protein + ( 1.2 \cdot g fiber )}{100 Calories} + {F}_{FD} + {F}_{EDen} - {F}_{Na} .$$

8. The method of claim 5, wherein the specific health concerns of said individual are a low-fat restriction and a low cholesterol restriction, and said individual wishes to:
a. maintain a healthy weight and the basic rating number for such individual is calculated by the following equation:
$$B 4 {B}_{3} = \frac{g protein + ( 1.2 \cdot g fiber )}{100 Calories} + {F}_{FD} - {F}_{Ch} - {F}_{Lipids} ;$$
or
b. lose weight while maintaining a healthy weight management plan and the basic rating number for such individual is calculated by the following equation:
$$B 4 {B}_{7} = \frac{g protein + ( 1.2 \cdot g fiber )}{100 Calories} + {F}_{FD} - {F}_{EDen} - {F}_{Ch} - {F}_{Lipids} ;$$
or
c. gain weight while maintaining a healthy weight management plan and the basic rating number for such individual is calculated by the following equation:
$$B 4 {B}_{11} = \frac{g protein + ( 1.2 \cdot g fiber )}{100 Calories} + {F}_{FD} + {F}_{EDen} - {F}_{Ch} - {F}_{Lipids} .$$

9. The method of claim 5, wherein the specific health concerns of said individual are a low-fat restriction, a low cholesterol restriction, and a low sodium restriction and said individual wishes to:
a. maintain a healthy weight and the basic rating number for such individual is calculated by the following equation:
$$B 4 {B}_{4} = \frac{g protein + ( 1.2 \cdot g fiber )}{100 Calories} + {F}_{FD} - {F}_{Ch} - {F}_{Lipids} - {F}_{Na} :$$
or
b. lose weight while maintaining a healthy weight management plan and the basic rating number for such individual is calculated by the following equation:
$$B 4 {B}_{8} = \frac{g protein + ( 1.2 \cdot g fiber )}{100 Calories} + {F}_{FD} - {F}_{Eden} - {F}_{Ch} - {F}_{Na} - {F}_{Lipids} :$$
or
c. gain weight while maintaining a healthy weight management plan and the basic rating number for such individual is calculated by the following equation:
$$B 4 {B}_{12} = \frac{g protein + ( 1.2 \cdot g fiber )}{100 Calories} + {F}_{FD} + {F}_{EDen} - {F}_{Na} - {F}_{Ch} - {F}_{Lipids} .$$

10-22. (canceled)

23. A method for rating the quality of food in a plurality of foods comprising:
calculating a single basic rating number for each of a first and second foods in said plurality of foods determined by the ratio derived from each food's energy, protein and fiber content and rating the food with the higher number as a better quality food than the food with a lower number and,
wherein the calculated number is raised or lowered as a function of one or more specific nutrients in the food, and
wherein the basic rating number is increased or decreased as a function of the presence of specific nutrients in the food in addition to protein and fiber that are related to specific health concerns of an individual, and
wherein said individual has no specific individual health concerns and wishes to:
a. maintain a healthy weight and the basic rating number for such individual is calculated by the following equation:
$$B 4 {B}_{1} = \frac{g protein + ( 1.2 \cdot g fiber )}{100 Calories} + {F}_{FD} ;$$
or
b. lose weight while maintaining a healthy weight management plan and the basic rating number for such individual is calculated by the following equation:
$$B 4 {B}_{5} = \frac{g protein + ( 1.2 \cdot g fiber )}{100 Calories} + {F}_{FD} - {F}_{EDen} ;$$
or
c. gain weight while maintaining a healthy weight management plan and the basic rating number for such individual is calculated by the following equation:
$$B 4 {B}_{9} = \frac{g protein + ( 1.2 \cdot g fiber )}{100 Calories} + {F}_{FD} - {F}_{EDen} .$$

24. A method for rating the quality of food in a plurality of foods comprising:
calculating a single basic rating number for each of a first and second foods in said plurality of foods determined by the ratio derived from each food's energy, protein and fiber content and rating the food with the higher number as a better quality food than the food with a lower number and,
wherein the calculated number is raised or lowered as a function of one or more specific nutrients in the food, and
wherein the basic rating number is increased or decreased as a function of the presence of specific nutrients in addition to protein and fiber that are related to specific health concerns of an individual, and
wherein the specific health concern of said individual is a low sodium restriction and said individual wishes to:
a. maintain a healthy weight and the basic rating number for such individual is calculated by the following equation:
$$B 4 {B}_{2} = \frac{g protein + ( 1.2 \cdot g fiber )}{100 Calories} + {F}_{FD} - {F}_{Na} ;$$
or
b. lose weight while maintaining a healthy weight management plan and the basic rating number for such individual is calculated by the following equation:
$$B 4 {B}_{6} = \frac{g protein + ( 1.2 \cdot g fiber )}{100 Calories} + {F}_{FD} - {F}_{EDen} - {F}_{Na} ;$$
or
c. gain weight while maintaining a healthy weight management plan and the basic rating number for such individual is calculated by the following equation:
$$B 4 {B}_{10} = \frac{g protein + ( 1.2 \cdot g fiber )}{100 Calories} + {F}_{FD} - {F}_{Ch} - {F}_{Lipids} - {F}_{Na} :$$

25. A method for rating the quality of food in a plurality of foods comprising:
calculating a single basic rating number for each of a first and second foods in said plurality of foods determined by the ratio derived from each food's energy, protein and fiber content and rating the food with the higher number as a better quality food than the food with a lower number, and
wherein the calculated number is raised or lowered as a function of one or more specific nutrients in the food, and
wherein the basic rating number is increased or decreased as a function of the presence of specific nutrients in addition to protein and fiber that are related to specific health concerns of an individual, and
wherein the specific health concerns of said individual are a low-fat restriction and a low cholesterol restriction, and said individual wishes to:
a. maintain a healthy weight and the basic rating number for such individual is calculated by the following equation:
$$B 4 {B}_{3} = \frac{g protein + ( 1.2 \cdot g fiber )}{100 Calories} + {F}_{FD} - {F}_{Ch} - {F}_{Lipids} ;$$
or
b. lose weight while maintaining a healthy weight management plan and the basic rating number for such individual is calculated by the following equation:
$$B 4 {B}_{7} = \frac{g protein + ( 1.2 \cdot g fiber )}{100 Calories} + {F}_{FD} - {F}_{EDen} - {F}_{Ch} - {F}_{Lipids} ;$$
or
c. gain weight while maintaining a healthy weight management plan and the basic rating number for such individual is calculated by the following equation:
$$B 4 {B}_{11} = \frac{g protein + ( 1.2 \cdot g fiber )}{100 Calories} + {F}_{FD} + {F}_{EDen} - {F}_{Ch} - {F}_{Lipids}$$

26. A method for rating the quality of food in a plurality of foods comprising:
calculating a single basic rating number for each of a first and second foods in said plurality of foods determined by the ratio derived from each food's energy, protein and fiber content and rating the food with the higher number as a better quality food than the food with a lower number, and
wherein the calculated number is raised or lowered as a function of one or more specific nutrients in the food, and
wherein the basic rating number is increased or decreased as a function of the presence of specific nutrients in addition to protein and fiber that are related to specific health concerns of an individual, and
wherein the specific health concerns of said individual are a low-fat restriction, a low cholesterol restriction, and a low sodium restriction and said individual wishes to:
a. maintain a healthy weight and the basic rating number for such individual is calculated by the following equation:
$$B 4 {B}_{4} = \frac{g protein + ( 1.2 \cdot g fiber )}{100 Calories} + {F}_{FD} - {F}_{Ch} - {F}_{Lipids} - {F}_{Na} ;$$
or
b. lose weight while maintaining a healthy weight management plan and the basic rating number for such individual is calculated by the following equation:
$$B 4 {B}_{8} = \frac{g protein + ( 1.2 \cdot g fiber )}{100 Calories} + {F}_{FD} - {F}_{Eden} - {F}_{Ch} - {F}_{Na} - {F}_{Lipids} ;$$
or
c. gain weight while maintaining a healthy weight management plan and the basic rating number for such individual is calculated by the following equation:
$$B 4 {B}_{12} = \frac{g protein + ( 1.2 \cdot g fiber )}{100 Calories} + {F}_{FD} + {F}_{EDen} - {F}_{Na} - {F}_{Ch} - {F}_{Lipids} .$$
